(19) United States
(12) Patent Application Publication
(10) Pub. No.: US 2008/0081841 A1
(43) Pub. Date: Apr. 3, 2008

(54) Materials and Methods for Modulating Cell Motility

(76) Inventors: Ali Badache, Marseille (FR); Nancy Hynes, Basel (CH); Romina Marone, Bellinzona (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/549,103

(22) PCT Filed: Mar. 18, 2004
(86) PCT No.: PCT/IB04/01280

§ 371 Date: Feb. 22, 2007

(30) Foreign Application Priority Data
Mar. 20, 2003 (GB) 0306464.9
Aug. 1, 2003 (GB) 0318126.0

(51) Int. Cl. A61K 35/00; A01K 67/02; A61P 43/00; C07K 14/00; C12N 1/00; C12N 5/02; C12P 21/00; G01N 33/53; C12Q 1/68; C12P 19/34; C12N 15/63; C07K 16/00; C07H 21/00; A01K 67/027

(52) U.S. Cl. 514/789; 435/243; 435/320.1; 435/375; 435/6; 435/69.1; 435/7.21; 435/91.1; 436/501; 530/350; 530/387.9; 536/23.1; 800/13; 800/21

(57) ABSTRACT

The invention is based on CGI-27 (named Memo for mediator of ErbB2-dependent cell motility) and its role in cell motility. The invention provides methods of inhibiting cell migration, particularly late phase cell migration e.g. which is induced by signals from the EGF-R. Also provided a related methods and materials for identifying and using inhibitors and other molecules, such as Memo binding partners, which may be used in modulating cell motility.

### FIELD OF THE INVENTION

The present invention concerns materials and methods relating to cell motility regulated by tyrosine kinase receptors.

### BACKGROUND

The Neu/ErbB2 gene encodes a 185-kDa transmembrane receptor tyrosine kinase that is a member of the epidermal growth factor receptor (EGFR) family. Heregulin (HRG) is a natural ligand of this receptor.

ErbB2 is often overexpressed in human tumors of diverse origins including breast and ovaries^{1,2}. Clinical studies have revealed that cancer patients whose tumors have alterations in ErbB2 expression tend to have more aggressive, metastatic disease, which is associated with parameters predicting a poor outcome³. In accordance with the clinical data, transgenic mice expressing activated Neu under the control of the mouse mammary tumor virus long terminal repeat develop metastatic mammary tumors⁴⁻⁶. Data from in vitro studies provide evidence that Neu/ErbB2 plays an important role in cancer cell motility and extracellular matrix invasion⁷⁻¹⁰. The molecular basis underlying ErbB2-dependent cell motility and metastases formation, however, remains poorly understood.

Activation of ErbB2 via dimerization with other ligand-bound ErbB members results in phosphorylation of tyrosine residues in the cytoplasmic tail^{11,12}. It is known that there is considerable functional redundancy in the autophosphorylation sites of Neu/ErbB2¹³. At least four of the five known sites can independently mediate transforming signals and can functionally substitute for each other. More recent work has suggested that in an activated Neu protein the tyrosine 1144 site has a role in stimulating metastasis, while the tyrosine 1227 site does not appear to be involved⁴.

The ErbB2 phosphotyrosines serve as high affinity binding sites for molecules containing Src homology 2 (SH2) or phosphotyrosine binding (PTB) domains such as the Shc and Grb2 adaptor molecules^{13,14} and the p85 subunit of phosphatidylinositol 3-kinase (PI3K)¹⁵. These docking proteins transduce proliferative, transforming or migratory signals to the cell nucleus via activation of, for example, the Ras/mitogen-activated protein kinase (MAPK) and the PI3K pathways¹⁶⁻¹⁹, both of which regulate different processes associated with cell migration, including formation of lamellipodia and actin stress fibers^{20,21}. There is also evidence that p38 kinase and c-Src induce actin reorganization via phosphorylation of focal adhesion proteins^{22,23}.

At the molecular level, some early studies indicated that Tyr 1144 binds Grb2 and Tyr 1201, 1227, and 1253 bind the Shc adaptor (Ricci et al., 1995, Oncogene, 11, 1519-1529). More recently, Crk was found to bind Tyr 1201 (Dankort et al., 2001 J Biol Chem. 276(42):38921-8.). All these pathways are believed to feed into the Ras/MAPK pathway.

Badache et al discussed a role for Y1201 and Y1227 of ErbB2 in regulating efficient cell migration in an abstract, published in the 2003 Proceedings of the AACR Online.

### DESCRIPTION OF INVENTION

The present inventors have characterised a protein known herein as MEMO (mediator of ErbB2-induced cell motility) as an important mediator of cell migration events.

The invention therefore relates to the newly identified mediator of cell migration and to methods of modulating it e.g. methods of inhibiting migration comprising inhibiting an activity of MEMO.

It further relates to assay methods for identifying factors which bind to or modulate the activity of MEMO, particularly factors which inhibit MEMO, and associated materials and methods.

No function has previously been ascribed to the MEMO polypeptide. A search by the inventors revealed that its nucleic acid sequence corresponds to that identified as CGI-27 in Lai et al., 2000, Genome Research 10:703-713. This document identified the predicted protein sequence as a hypothetical protein only. Moreover, its sequence does not provide any information as to its potential role. Surprisingly, the protein does not contain a SH2 or PTB domain, which are known to bind to phosphotyrosines.

The present inventors have determined that MEMO is a mediator of ErbB2 signalling, particularly signalling from Y1227 of ErbB2. Reduction of MEMO's activity using siRNA resulted in a reduction, upon phosphorylation of this site, of heregulin-induced migration in cells having a tyrosine at position 1227 but a Phe residue at position 1201 of ErbB2. No such reduction is seen in cells having a tyrosine residue at position 1201 and a Phe at position 1227. This contrasts for example to the effect seen upon downregulation of Shc and Crk, where a reduction of either of these activities inhibits motility in both cell types.

In addition, the inventors have found that inhibiting the activity of MEMO causes a cellular response which has not previously been observed for a signalling molecule acting downstream of a tyrosine kinase receptor. When factors such as Shc, Crk or phospholipase Cγ1 are downregulated or inhibited, the cells fail to undergo even the preliminary morphological changes of the migration process. In contrast, when MEMO is inhibited, the cells are able to undergo initial morphological changes, but still fail to show sustained cell motility.

Accordingly, it is believed that MEMO acts in the late effect pathway downstream of Y1227.

It is believed that the MEMO-dependent pathway may be a useful therapeutic target. In particular it is believed that it may in preferred embodiments provide the basis for more specific regulators of cell motility than the pathways which have been previously implicated in migration, and which are known to be involved in the regulation of many other cellular processes, such as proliferation, differentiation, inflammation and survival.

Surprisingly, under the experimental conditions used, reduction of MEMO's activity in cells expressing wild type ErbB2/Neu reduced heregulin-induced cell migration by 50%. This was unexpected because of the functional redundancy that has been observed in the tyrosine autophosphorylation sites of ErbB2, and suggests that the pathway in which MEMO acts may be a useful therapeutic target either alone or in combination with others (e.g., the pathway initiated by Y1201 phosphorylation).

These and other aspects of the present invention will now be discussed in more detail:

In a first embodiment, the invention provides a method of modulating, e.g., inhibiting cell migration, comprising modulating, e.g., inhibiting an activity of MEMO. For example, inhibition may be of the cell migration which occurs in response to a migration-inducing signal, e.g., in a tumour cell or a cell implicated in cancer or a metastatic disease.

Cell “migration” or “motility” can be viewed as a series of morphological changes based on remodelling of the cytoskeleton. After receiving a migration-inducing signal, a cell undergoes a number of changes including lamellipodia formation. Initially the lamellipodia extend in all directions, before showing a more polar organisation, reflecting the formation of actin stress fibres. This is followed by formation of a connection to the substratum via a focal adhesion, and then by movement of the whole cell relative to the substratum. Cell migration or motility refers to this process as a whole, and to the outcome thereof which is the movement of a cell from one location to another in its surrounding environment, particularly relative to the substratum.

Inhibition of cell migration as used herein is intended, unless the context demands otherwise, to refer to inhibition of any stage of the cell motility process such that when a cell receives a signal (known herein as a “migration inducing signal”) which would in the absence of said inhibition cause it to undergo said stage, the stage is not completed. The result of this will be inhibition of movement of the cell. For example, movement may be at a slower rate or for a shorter period or may not occur at all in at least some cells.

A migration-inducing signal may for example be a signal received from the ErbB2 receptor. In addition, the inventors have also found that downregulating MEMO affects late-stage migration in cells that have been stimulated by FGF2 or EGF. Accordingly, a migration-inducing signal may also be a signal received from other tyrosine kinase receptors such as the Fibroblast Growth factor (FGF) 2 or Epidermal Growth Factor (EGF) receptors. The signal may be received constitutively, e.g., as a result of over-expression and/or constitutive activity of the receptor. Alternatively, it may be received when the receptor is contacted with a ligand, such as heregulin, EGF, amphiregulin, TGF alpha, FGF, or an artificial ligand.

Inhibition of an “activity” of MEMO is used broadly in this aspect to encompass any situation in which the effectiveness of MEMO in the pathway which positively regulates motility in response to a signal is reduced e.g. by down regulating MEMO, or inhibiting any positive interaction with other members of the pathway such as its binding partners.

Preferably, the inhibition is specific to MEMO in the sense that it does not appreciably inhibit the activity of other factors e.g. those not implicated in migration. Preferably, it does not appreciably affect the activity of a factor which is a mediator of early stage migration as explained further below. Most preferably, it does not appreciably affect the activity of any factor which is not downstream of (and e.g. activated by) MEMO.

For example the activity MEMO may be inhibited by inhibiting transcription and/or translation of MEMO. In examples described herein siRNAs were used for this purpose, but other methods of specifically down-regulating the expression of particular genes will be well known to those skilled in the art e.g. the use of ribozymes (see e.g. Jaeger (1997) The new world of ribozymes, Curr Opin Struct Biol 7:324-335, or Gibson & Shillitoe (1997)Ribozymes: their functions and strategies form their use, Mol Biotechnol 7: 242-251.)

Alternatively, the inhibition may be post-translational. In this embodiment, the inhibitor may for example be a small molecule, an antibody or antibody fragment or a polypeptide. Methods of producing antibodies against MEMO, and identifying inhibitors, are discussed hereinafter.

In particular, the inhibitor may, for example, inhibit the binding of MEMO with its natural binding partner, which may for example be an upstream or downstream factor. This may thus prevent its modulation (e.g. activation) by or of these factors.

The invention further provides Memo-based methods of modulating microtubule outgrowth, for example ErbB2-dependent elongation of microtubules, to the cell cortex or periphery from the centrosome.

MEMO has been identified by the present inventors as a mediator of inter alia ErbB2-induced motility. Thus in a still preferred embodiment, the activity of MEMO is inhibited e.g. by inhibiting its binding to and/or activation by ErbB2 e.g. phosphorylated Y1227 of ErbB2.

In this embodiment, the inhibitor may be an antibody, small molecule or polypeptide fragment which binds specifically to MEMO or to ErbB2 (e.g. to a site comprising or proximal to Y1227 of ErbB2), and which physically inhibits MEMO binding to and/or activation following phosphorylation of Y1227 of ErbB2. Preferably, it binds specifically to MEMO. Such inhibitors can be provided as described below.

The cytoskeleton re-modelling as discussed above is widely used as a way of assessing cell migration. However, the present inventors have identified a late stage pathway which affects sustained cell motility but which does not affect early morphological changes in the cell. Cells in which signalling from Y1201/Y1227 of ErbB2 (i.e. signalling resulting from phosphorylation of these sites) is inhibited, or in which MEMO is down regulated, show normal lamellipodia formation, actin cytoskeleton organisation and lamellipodia organisation at least as an initial response to a migration-inducing signal. However, after a time, these cellular responses are reduced, and cell migration is inhibited.

Thus, in a preferred embodiment, inhibition of activity is used to inhibit a late stage of migration. Preferably the inhibition of activity is used to inhibit a late stage in preference to an early stage of migration.

A “late stage” of cell migration is therefore a stage of the cell migration process which is required to sustain cell migration in response to a migration-inducing signal.

The present inventors have found that the late-stage migration is dependent on de novo protein synthesis. Accordingly, whether a stage is transcription and/or translation dependent can be used to assess whether it is a late-stage. Early stage migration effects are not dependent on de novo protein synthesis, while late stage migration effects are so dependent.

Generally speaking, it is believed that late stage events follow initial lamellipodia formation (i.e., the earliest onset of lamellipodia formation which is observable in response to a migration-inducing signal). Preferably, they also follow initial lamellipodia organisation (i.e., the earliest onset of lamellipodia organisation which is observable after the cell receives a migration-inducing signal). Accordingly, when a late stage is inhibited, at least initial lamellipodia formation will generally still be observed in response to a migration-inducing signal, and preferably also initial lamellipodia organisation.

Accordingly an “early stage” of cell migration is a stage which precedes late stage. Initial lamellipodia formation is, for example, generally considered to be an early-stage effect.

For example, one suitable test for assessing whether a late or early stage has been inhibited in a given cell may be to compare the response of said cell with the responses of a second cell in which de novo protein synthesis has been inhibited. The response is a response to a migration inducing signal. If the two cells show a similar response to the migration-inducing signal then the stage can be designated as a late stage of migration. If however the cell in which de novo protein synthesis is inhibited is able to complete more stages of the migration process, then the stage can be designated as an early stage.

Whether a stage is late or early can also be assessed by the time it occurs after the migration-inducing signal using visualisation or other techniques well known to those skilled in the art, and demonstrated in the Examples below. In the present examples, events happening in at least the first 30 minutes after the migration-inducing signal were considered to be early stage, though the exact timing will differ depending on the experimental conditions.

It is also believed that MEMO acts more specifically in migration than those factors previously implicated in migration, which are implicated in the regulation of early cellular events and which are know to be involved in other processes, e.g., proliferation and survival. For instance, the present inventors have found that cells transfected with siRNA to MEMO are viable over several days, and also that MEMO does not appear to be required for proliferation of SKBr3 cells.

Methods of inhibiting cell migration as described above (i.e. for inhibiting a late stage in preference to an early stage of migration) may be of therapeutic value, e.g., in preventing metastasis and angiogenesis in cancer, and in preventing inflammation (caused by migration of macrophages and other cells of the immune system). It may also be useful for example in preventing scar tissue accumulation.

Although the discussion above has focused on inhibition of cell migration, the invention also provides in an another aspect methods of enhancing cell migration, which may for example be useful in promoting wound healing. Such methods may comprise providing MEMO (including variants and fragments of MEMO as defined below) or an activator of MEMO to a cell. Accordingly, in a another aspect there is provided use of MEMO to promote wound healing.

As can be seen from the above, the present inventors have identified MEMO as an important mediator of late-stage cell migration events.

Thus in one aspect of the invention, there is provided a method for identifying substances which bind to and/or modulate the activity of MEMO. Preferably, the assay method is for the identification of substances which bind to and/or inhibit the activity of MEMO. It is envisaged that such substances be used in methods of treatment.

More preferably, the substances also inhibit cell migration, and may be used for example in methods of treatment where it is desired to inhibit migration.

Accordingly, the methods may include the further step of confirming whether the same substance inhibits cell migration, and particularly if it inhibits cell migration without affecting early stage cell migration events as described below.

As those skilled in the art will appreciate, unless context demands otherwise, where polypeptides or nucleic acids are referred to in aspects and embodiments of the invention disclosed herein (e.g. ErbB2, MEMO) variants (e.g. derivatives or homologues) of the polypeptides or nucleic acids specified above may also be used in the present invention, provided that they still encode the requisite activity. For example where reference to ErB2 is made, embodiments of the invention will embrace the use of Neu, unless context demands otherwise. Generally speaking such variants will be substantially homologous to the ‘wild type’ or other sequence specified herein i.e. will share sequence similarity or identity therewith. Similarity or identity may be at the nucleotide sequence and/or encoded amino acid sequence level, and will preferably, be at least about 50%, 60%, or 70%, or 80%, most preferably at least about 90%, 95%, 96%, 97%, 98% or 99%. Sequence comparisons may be made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA. Analysis for similarity can also be carried out using hybridisation. One common formula for calculating the stringency conditions required to achieve hybridization between nucleic acid molecules of a specified sequence homology is: Tₘ=81.5° C.+16.6 Log [Na+]+0.41 (% G+C)−0.63 (% formamide)−600/#bp in duplex (Molecular Cloning: a Laboratory Manual: 2nd edition, Sambrook et al., 1989, Cold Spring Harbor Laboratory Press).

Preferred fragments and variants of MEMO are as described below, and in particular are functional fragments and variants, preferably those which retain the ability to mediate migration events, more preferably tyrosine kinase induced events, still more preferably those induced by ErbB2. Preferably the activity which is retained is the ability to mediate late-stage migration events without mediating early stage events.

Binding assays may be competitive or non-competitive.

Assays will be run with suitable controls routine to those of skill in the art.

In this and other aspects, putative or actual inhibitors or other modulators may be provided from any source which it is desired to screen, and may or may not be naturally occurring or synthetic, and may or may not be peptides or polypeptides (e.g. antibodies) or nucleic acids (e.g. siRNA). Preferred inhibitors most suited for therapeutic applications will be small molecules e.g. from a combinatorial library such as are now well known in the art (see e.g. Newton (1997) Expert Opinion Therapeutic Patents, 7(10): 1183-1194). Preferred candidate substances may include small molecules such as those of the steroid, benzodiazepine or opiate classes.

In one embodiment the invention provides a method which comprises the step of contacting a cell expressing MEMO with a test substance and identifying substances which inhibit the activity of MEMO in the cell.

For example, the present inventors have found that MEMO is involved in a late-stage of migration which is dependent on de novo protein synthesis (or gene transcription). It is believed that MEMO might contribute to transcriptional activation of a specific set of genes which are essential for late-stage cell migration. Accordingly, assays of the invention may be conducted by utilizing the ability of MEMO to activate such genes, and the ability of inhibitors to inhibit that process.

In another embodiment of the invention the inhibition of the interaction of MEMO with a binding partner is assessed. This may comprise (i) contacting MEMO with a binding partner thereof in the presence and absence of a test substance; and

(ii) determining whether the presence of a test substance inhibits the interaction between MEMO and its binding partner.

Methods for assessing the interaction between a polypeptide and a binding partner may be any of the methods known to those skilled in the art and are disclosed here. Any of these methods can be used to assess whether a test substance inhibits the interaction between a polypeptide (in this case MEMO) and a binding partner.

In one embodiment, assays are those based upon MEMO and its interaction with upstream factors such as Erb2, in particular with phosphorylated Y1227 of ErbB2.

One embodiment of this aspect may comprise:

(i) providing a polypeptide which is ErbB2, or a fragment thereof, comprising a phosphorylated residue corresponding to Y1227;
(ii) contacting said polypeptide with MEMO, in the presence and absence of a test substance; and
(iii) determining whether the presence or absence of a test substance inhibits the interaction between the polypeptide and MEMO.

A residue “corresponding to” Y1227 of ErbB2 is a residue in the same amino acid environment. In particular it is an acidic residue, and still more preferably it is a phosphotyrosine residue. Whether the residue is in the same amino acid environment as Y1227 of ErbB2 can be assessed for example by aligning the portion of the polypeptide containing the residue of interest with the portion of ErbB2 containing Y1227. If the sequence is identical over at least 5 amino acids, preferably over at least 10 or 16 amino acids, then the residues can be said to be in a corresponding environment.

Preferably the fragment comprises a residue corresponding to Y1227 but does not comprise any other phosphorylated residue (termed herein a YC polypeptide).

Still more preferably, the YC polypeptide is a fragment which does not contain any phosphorylation site (i.e. residue susceptible to phosphorylation) other than Y1227. This allows the residue corresponding to Y1227 to be phosphorylated during synthesis without the need to mask any other potential phosphorylation sites. Preferably such fragments will comprise an amino acid sequence which is identical to a portion of the amino acid sequence of ErbB2 at least 5, preferably at least 10 or 16 amino acids amino acids in length, and which comprises phosphorylated Y1227.

Determining whether the test substance inhibits the interaction between MEMO and an ErbB2 polypeptide may comprise determining whether the activation of MEMO is inhibited. For example, the activation may involve chemical modification (e.g., phosphorylation). This may be detected for example by using phospho-specific antibodies, by looking for incorporation of radiolabelled phosphate, or using phosphopeptide mapping.

In an alternative, determining whether the test substance inhibits the interaction between MEMO and an ErbB2 polypeptide may comprise determining whether the physical association between the ErbB2 polypeptide and MEMO is inhibited. This may be achieved as described hereinafter.

As discussed below, the association between MEMO and its binding partner may be via an “adaptor” molecule, such as those discussed in ref[4]. Thus in another embodiment, assays are those based upon MEMO and its interaction with shc, which is described in the Examples below.

In another embodiment, the interaction may be between MEMO and a downstream binding partner. The downstream binding partner may for example be provided by the methods described herein below. Preferably, determining whether the test substance inhibits the interaction between MEMO and a downstream binding partner comprises determining whether the physical interaction is inhibited, but it may comprise determining whether activation of the downstream factor is inhibited, for example, as discussed above.

Assays according to the invention may be performed in vitro. For example, the physical association between MEMO and a binding partner thereof may be studied by labelling one with a detectable label and bringing it into contact with the other which has been immobilised on a solid support. Suitable detectable labels include ³⁵S-methionine which may be incorporated into recombinantly produced MEMO and/or the binding partner thereof. The recombinantly produced MEMO and/or binding partner may also be expressed as a fusion protein containing an epitope which can be labelled with an antibody. Alternatively, double-labelling may be used as is well known in the art, for example, using a radioactive label and a scintillant.

Generally, a protein which is immobilized on a solid support may be immobilized using an antibody against that protein bound to a solid support or via other technologies which are known per se. A preferred in vitro interaction may utilise a fusion protein including a tag, such as glutathione-S-transferase (GST) or His6. The tag may be immobilized by affinity interaction, for example on glutathione agarose beads or Ni-matrices, respectively.

In an in vitro assay format of the type described above the putative inhibitor compound can be assayed by determining its ability to modulate the amount of labelled MEMO or binding partner which binds to the immobilized binding partner, e.g., GST-binding partner or GST-MEMO as the case may be. This may be determined by fractionating the glutathione-agarose beads by SDS-polyacrylamide gel electrophoresis. Alternatively, the beads may be rinsed to remove unbound protein and the amount of protein which has bound can be determined by counting the amount of label present in, for example, a suitable scintillation counter.

Alternatively an antibody attached to a solid support and directed against one of MEMO or the binding partner may be used in place of GST to attach the molecule to the solid support. Antibodies against MEMO and its binding partners may be obtained in a variety of ways known as such in the art, and as discussed herein.

In an alternative mode, one of MEMO and its binding partner may be labelled with a fluorescent donor moiety and the other labelled with an acceptor which is capable of reducing the emission from the donor. This allows an assay according to the invention to be conducted by fluorescence resonance energy transfer (FRET). In this mode, the fluorescence signal of the donor will be altered when MEMO and its binding partner interact. The presence to a candidate modulator compound which modulates the interaction will increase the amount of unaltered fluorescence signal of the donor.

FRET is a technique known per se in the art and thus the precise donor and acceptor molecules and the means by which they are linked to MEMO and its binding partner may be accomplished by reference to the literature.

Suitable fluorescent donor moieties are those capable of transferring fluorogenic energy to another fluorogenic molecule or part of a compound and include, but are not limited to, coumarins and related dyes such as fluoresceins, rhodols and rhodamines, resorufins, cyanine dyes, bimanes, acridines, isoindoles, dansyl dyes, aminophthalic hydrazines such as luminol and isoluminol derivatives, aminophthalimides, aminonaphthalimides, aminobenzofurans, aminoquinolines, dicyanohydroquinones, and europium and terbium complexes and related compounds.

Suitable acceptors include, but are not limited to, coumarins and related fluorophores, xanthenes such as fluoresceins, rhodols and rhodamines, resorufins, cyanines, difluoroboradiazaindacenes, and phthalocyanines.

A preferred donor is fluorescein and preferred acceptors include rhodamine and carbocyanine. The isothiocyanate derivatives of these fluorescein and rhodamine, available from Aldrich Chemical Company Ltd, Gillingham, Dorset, UK, may be used to label MEMO and its binding partner. For attachment of carbocyanine, see for example Guo et al, J. Biol. Chem., 270; 27562-8, 1995.

Assays of the invention may also be performed in vivo. Such an assay may be performed in any suitable host cell, e.g. a bacterial, yeast, insect or mammalian host cell. Yeast and mammalian host cells are particularly suitable.

To perform such an assay in vivo, constructs capable of expressing MEMO and its binding partner and a reporter gene construct may be introduced into the cells. This may be accomplished by any suitable technique, for example calcium phosphate precipitation or electroporation. The constructs may be expressed transiently or as stable episomes, or integrated into the genome of the host cell.

In vivo assays may also take the form of two-hybrid assays. Two-hybrid assays may be in accordance with those disclosed by Fields and Song, 1989, Nature 340; 245-246. In such an assay the DNA binding domain (DBD) and the transcriptional activation domain (TAD) of the yeast GAL4 transcription factor are fused to the first and second molecules respectively whose interaction is to be investigated. A functional GAL4 transcription factor is restored only when two molecules of interest interact. Thus, interaction of the molecules may be measured by the use of a reporter gene operably linked to a GAL4 DNA binding site which is capable of activating transcription of said reporter gene. Other transcriptional activator domains may be used in place of the GAL4 TAD, for example the viral VP16 activation domain.

In general, MEMO and its binding partner are expressed as fusion proteins, one being a fusion protein comprising a DNA binding domain (DBD), such as the yeast GAL4 binding domain, and the other being a fusion protein comprising an activation domain, such as that from GAL4 or VP16. In such a case the host cell (which again may be bacterial, yeast, insect or mammalian, particularly yeast or mammalian) will carry a reporter gene construct with a promoter comprising a DNA binding elements compatible with the DBD.

MEMO and its binding partner and the reporter gene, may be introduced into the cell and expressed transiently or stably.

MEMO and/or its binding partner may then be contacted with a test substance, and inhibition of binding between MEMO and its binding partner can be observed as a reduction of reporter gene expression.

Inhibitors identified in this screen may for example be used to inhibit cell migration events, as described above. They may be formulated as medicaments as described hereinafter.

In some embodiments, e.g., in methods involving assessment of the interaction between MEMO and a downstream factor, it is preferred that MEMO is in its activated form. For example, for in vitro methods, activated MEMO may be provided by immunoprecipitation of MEMO or affinity purification of tagged MEMO from an activated cell-sample. The cell may be a cell that has been stimulated with heregulin. For in vivo methods, it may for example be achieved by performing a method in a cell which expresses a tyrosine kinase receptor such as ErbB2, e.g., by also expressing said tyrosine kinase receptor in the cell. The receptor may be activated by the provision of a ligand. Alternatively, it may be constitutively active.

The method may optionally further comprise a functional assay, to confirm whether the inhibitor of MEMO activity identified as above is a mediator of cell migration.

In one embodiment, the method comprises the step of contacting a cell with the inhibitor of MEMO activity, and confirming that the inhibitor is capable of inhibiting cell motility. For example, the inhibition of cell motility may be of cell motility which occurs in response to a migration-inducing signal, as discussed above.

The cells used in this method may be cells which constitutively show a high degree of cell migration, for example, MDA-MB-231 cells. Alternatively, the method may comprise contacting the cell with a factor e.g. a ligand which stimulates cell migration.

Preferably the method is for identifying an inhibitor of ErbB2 induced cell migration events (particularly induced by phosphorylated Y1201 or Y1227). In this embodiment, the method may comprise contacting the cell with a ligand for ErbB2, for example the natural ligand heregulin, or may comprise using a cell expressing ErbB2 which is constitutively active.

A suitable assay for migration may be any of the assays known in the art, for example Transwell-type assays in 96-well format or measure of cell motility using Cellomics-type High Content Screen for Cell Motility, as shown in the Examples.

Preferably, method comprises determining whether the test substance is an inhibitor of late, but not (or at least in preference to) early stage migration events. Methods are disclosed hereinbelow.

As explained above, in one embodiment this may comprise identifying substances which inhibit cell migration but which do not (significantly) inhibit initial lamellipodia formation.

Accordingly, the method may comprise assaying for an effect on migration, as above, and then

(ii) among these inhibitors, screening for inhibitors not affecting lamellipodia formation (this can be measured by evaluating cell spreading using tools such as Cellomics ArrayScan system for Cell Spreading).

The invention further provides materials and methods which have utility in performing various aspects of the invention.

Accordingly, the present invention provides an isolated MEMO polypeptide comprising the amino acid sequence shown in Genbank AF132961. The nucleotide sequence and predicted translated sequence is shown in the Sequence Annex attached hereto.

Isolated MEMO polypeptides of the invention will be those as defined above in isolated form, free or substantially free of material with which it is naturally associated such as other polypeptides with which it is found in the cell. The polypeptides may be modified, e.g., glycosylated, either naturally or by systems of heterologous eukaryotic cells, or they may be (for example if produced by expression in a prokaryotic cell) unglycosylated. Polypeptides may be phosphorylated and/or acetylated.

A polypeptide of the invention may also be in a substantially purified form, in which case it will generally comprise the polypeptide in a preparation in which more than 90%, e.g. 95%, 98% or 99% of the polypeptide in the preparation is a polypeptide of the invention.

MEMO polypeptides of the invention may be modified for example by the addition of histidine residues to assist their purification or by the addition of a signal sequence to promote their secretion from a cell. All or part of the MEMO polypeptides of the invention may also be expressed as fusion proteins e.g. for use in yeast two hybrid systems.

Thus as discussed hereinbefore, the invention also provides for isolated polypeptides which are variants of MEMO, having e.g. at least 50%, 60%, 70%, 80%, 90%, 95% or 99% amino acid sequence identity thereto. Preferably, the variant retains at least one function of MEMO having the sequence of AF132961, preferably the ability to mediate cell migration, more preferably migration induced by tyrosine kinase receptors, preferably ErbB2, and still more preferably by phosphorylation of Y1227 of ErbB2. Preferably the migration that is mediated is late stage and not early stage migration.

The term variants also includes naturally occurring alleles, orthologs and other homologs of the MEMO sequence shown herein.

There is further provided an isolated polypeptide which is a fragment of the polypeptide as shown in AF132961, said fragment being at least 10, for example at least 20, 30, 40, 50, 75, 100 or 150 or more amino acids in size. Preferably the fragment retains a function of MEMO as above.

Variants and fragments may include the DUF52 domain, which has been identified in the MEMO sequence. This domain is shared by several proteins of unknown function in species including yeast, C. elegans, drosophila and mouse.

It will be understood that references to MEMO in the present application include references to these fragments and variants, and in particular to functional fragments and variants.

A polypeptide according to the present invention may be used as an immunogen or otherwise in obtaining specific antibodies. Antibodies are useful in purification and other manipulation of polypeptides, diagnostic screening and therapeutic contexts. This is discussed further below.

A polypeptide according to the present invention may be used in screening for molecules which bind to it or modulate its activity or function. Such molecules may be useful in a therapeutic (possibly including prophylactic) context. This is discussed further below.

In a further aspect, the invention provides a vector comprising a polynucleotide comprising a nucleic acid sequence encoding MEMO or a variant or fragment thereof as above.

The vectors will be recombinant replicable vectors, and may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

The vectors are preferably expression vectors comprising a promoter operably linked to said nucleic acid sequence. The vectors may be carried by a host cell, and expressed within said cell. Following said expression, polypeptides of the invention may be recovered.

The term “operably linked” refers to a juxtaposition wherein the components described are in a relationship permitting them to function in their intended manner. A control sequence “operably linked” to a coding sequence is ligated in such a way that expression of the coding sequence is achieved under condition compatible with the control sequences.

Suitable vectors can be chosen or constructed, containing appropriate regulatory sequences, including promoter sequences, terminator fragments, polyadenylation sequences, enhancer sequences, marker genes and other sequences as appropriate.

Vectors may be plasmids, viral e.g. ‘phage phagemid or baculoviral, cosmids, YACs, BACs, or PACs as appropriate. Vectors include gene therapy vectors, for example vectors based on adenovirus, adeno-associated virus, retrovirus (such as HIV or MLV) or alpha virus vectors.

The vectors may be provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo, for example in methods of gene therapy. Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others.

Promoters and other expression regulation signals may be selected to be compatible with the host cell for which the expression vector is designed. For example, yeast promoters include S. cerevisiae GAL4 and ADH promoters, S. pombe nmt1 and adh promoter. Mammalian promoters include the metallothionein promoter which is responsive to heavy metals such as cadmium. Viral promoters such as the SV40 large T antigen promoter or adenovirus promoters may also be used. All these promoters are readily available in the art.

The vectors may include other sequences such as promoters or enhancers to drive the expression of the inserted nucleic acid, nucleic acid sequences so that the polypeptide is produced as a fusion and/or nucleic acid encoding secretion signals so that the polypeptide produced in the host cell is secreted from the cell. Vectors for production of polypeptides of the invention or for use in gene therapy include vectors which carry a mini-gene sequence of the invention.

For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Vectors may be transformed into a suitable host cell as described above to provide for expression of a polypeptide of the invention. Thus, in a further aspect the invention provides a process for preparing polypeptides according to the invention which comprises cultivating a host cell transformed or transfected with an expression vector as described above under conditions to provide for expression by the vector of a coding sequence encoding the polypeptides, and recovering the expressed polypeptides. Polypeptides may also be expressed in in vitro systems, such as reticulocyte lysate.

A further embodiment of the invention provides host cells transformed or transfected with the vectors for the replication and expression of polynucleotides of the invention. The cells will be chosen to be compatible with the said vector and may for example be bacterial, yeast, insect or mammalian.

Most of the foregoing has been concerned with the expression of sequences encoding MEMO or variants thereof in order to increase MEMO activity in a cell. However in other aspects the invention relates to methods and materials for reducing MEMO activity in a cell e.g. by pre- or post-transcriptional silencing.

Thus polynucleotides according to the invention include those in which the complement of the MEMO coding sequence is included. Thus MEMO may also be inserted into the vectors described above in an antisense orientation in order to provide for the production of antisense RNA or ribozymes.

An alternative to anti-sense is to use double stranded RNA (dsRNA) which has been found to be even more effective in gene silencing than both sense or antisense strands alone (Fire A. et al Nature, Vol 391, (1998)). dsRNA mediated silencing is gene specific and is often termed RNA interference (RNAi) (See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245).

RNA interference is a two step process. First, dsRNA is cleaved within the cell to yield short interfering RNAs (siRNAs) of about 1-23 nt length with 5′ terminal phosphate and 3′ short overhangs (˜2 nt) The siRNAs target the corresponding mRNA sequence specifically for destruction (Zamore P. D. Nature Structural Biology, 8, 9, 746-750, (2001).

Thus in one embodiment, the invention provides double stranded RNA comprising a MEMO-encoding sequence, which may for example be a “long” double stranded RNA (which will be processed to siRNA, e.g., using Dicer). These RNA products may be synthesised in vitro, e.g., by conventional chemical synthesis methods.

RNAi may be also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)).

Thus siRNA duplexes containing between 20 and 25 bps, more preferably between 21 and 23 bps, of the MEMO sequence form one aspect of the invention e.g. as produced synthetically, optionally in protected form to prevent degradation.

Alternatively siRNA may be produced from a vector, in vitro (for recovery and use) or in vivo.

Accordingly, the vector may comprise a nucleic acid sequence encoding MEMO (including a nucleic acid sequence encoding a variant or fragment thereof), suitable for introducing an siRNA into the cell in any of the ways known in the art, for example, as described in any of references cited herein, which references are specifically incorporated herein by reference.

In one embodiment, the vector may comprise a nucleic acid sequence according to the invention in both the sense and antisense orientation, such that when expressed as RNA the sense and antisense sections will associate to form a double stranded RNA. This may for example be a long double stranded RNA (e.g., more than 23 nts) which may be processed in vitro with Dicer to produce siRNAs (see for example Myers (2003) Nature Biotechnology 21:324-328) or siRNA hairpin structures.

Alternatively, the double stranded RNA may directly encode the sequences which form the siRNA duplex, as described above. In another embodiment, the sense and antisense sequences are provided on different vectors.

These vectors and RNA products may be useful for example to inhibit de novo production of the MEMO polypeptide in a cell. They may be used analogously to the expression vectors in the various embodiments of the invention discussed herein.

A still further aspect of the present invention provides a method which includes introducing the nucleic acid (e.g. any of the vectors discussed above) into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. As an alternative, direct injection of the nucleic acid could be employed.

The introduction may be followed by causing or allowing transcription, and where appropriate expression, from the nucleic acid, e.g. by culturing host cells (which may include cells actually transformed although more likely the cells will be descendants of the transformed cells) under conditions for expression of the gene, so that the encoded polypeptide or RNA molecule is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers (e.g. see below).

A further aspect of the present invention provides a host cell containing nucleic acid as disclosed herein. The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

In another embodiment, the invention provides a transgenic animal in which expression of MEMO is modified.

In another aspect of the invention, there is provided a method for producing a transgenic non-human mammal, particularly a rodent such as a mouse, by incorporating a lesion into the locus of a MEMO gene.

This may be achieved in a variety of ways. A typical strategy is to use targeted homologous recombination to replace, modify or delete the wild-type MEMO gene in an embryonic stem (ES) cell. A targeting vector is introduced into ES cells by electroporation, lipofection or microinjection. In a few ES cells, the targeting vector pairs with the cognate chromosomal DNA sequence and transfers the desired mutation carried by the vector into the genome by homologous recombination. Screening or enrichment procedures are used to identify the transfected cells, and a transfected cell is cloned and maintained as a pure population. Next, the altered ES cells are injected into the blastocyst of a preimplantation mouse embryo or alternatively an aggregation chimera is prepared in which the ES cells are placed between two blastocysts which, with the ES cells, merge to form a single chimeric blastocyst. The chimeric blastocyst is surgically transferred into the uterus of a foster mother where the development is allowed to progress to term. The resulting animal will be a chimera of normal and donor cells. Typically the donor cells will be from an animal with a clearly distinguishable phenotype such as skin colour, so that the chimeric progeny is easily identified. The progeny is then bred and its descendants cross-bred, giving rise to heterozygotes and homozygotes for the targeted mutation. The production of transgenic animals is described further by Capecchi, M, R., 1989, Science 244; 1288-1292; Valancius and Smithies, 1991, Mol. Cell. Biol. 11; 1402-1408; and Hasty et al, 1991, Nature 350; 243-246, the disclosures of which are incorporated herein by reference.

There are also provided transgenic animals in which the downregulation of MEMO is conditional. For example, this allows for the animal to develop if the lesion of the MEMO gene is embryonic lethal. This may involve for example providing an inhibitor of MEMO, e.g., an siRNA or an antisense RNA, under the control of an expression system as described above, wherein the expression of the inhibitor can be induced conditionally at an appropriate developmental stage e.g. by placing it under the control of an inducible promoter and applying an appropriate stimulus.

Homologous recombination in gene targeting may be used to replace the wild-type MEMO gene with a specifically defined mutant form (e.g. truncated or containing one or more substitutions).

The invention may also be used to replace the wild-type gene with a modified gene capable of expressing a wild-type or otherwise active MEMO polypeptide, where the expression may be selectively blocked either permanently or temporarily. Permanent blocking may be achieved by supplying means to delete the gene in response to a signal. An example of such a means is the cre-lox system where phage lox sites are provided at either end of the transgene, or at least between a sufficient portion thereof (e.g. in two exons located either side of one or more introns). Expression of a cre recombinase causes excision and circularisation of the nucleic acid between the two lox sites. Various lines of transgenic animals, particularly mice, are currently available in the art which express cre recombinase in a developmentally or tissue restricted manner, see for example Tsien, Cell, Vol. 87(7): 1317-1326, (1996) and Betz, Current Biology, Vol. 6(10): 1307-1316 (1996). These animals may be crossed with LoX transgenic animals of the invention to examine the function of the MEMO gene. An alternative mechanism of control is to supply a promoter from a tetracyline resistance gene, tet, to the control regions of the MEMO locus such that addition of tetracyline to a cell binds to the promoter and blocks expression of the MEMO gene.

Transgenic targeting techniques may also be used to delete the MEMO gene. Methods of targeted gene deletion are described by Brenner et al, WO94/21787 (Cell Genesys), the disclosure of which is incorporated herein by reference.

Homologous recombination may also be used to produce “knock in” animals which express a polypeptide of the invention in the form of a fusion protein, fused to a detectable tag such as β-galactosidase or green fluorescent protein. Such transgenic non-human mammals may be used in methods of determining temporal and spatial expression of the MEMO gene by monitoring the expression of the detectable tag.

A further alternative is to target control sequences responsible for expression of the MEMO gene.

The invention extends to transgenic non-human mammals obtainable by such methods and to their progeny. Such mammals may be homozygous or heterozygous. Such mammals include mice, rodents, rabbits, sheep, goats, and pigs.

Transgenic non-human mammals may be used for experimental purposes e.g. in studying the role of MEMO in regulating cell migration and in the development of therapies designed to target the interaction of MEMO with other cellular factors, particularly those involved in metastases and other instances where cell migration is undesirable, or in cases where cell migration may be desirable e.g. wound healing. By “experimental” it is meant permissible for use in animal experimentation or testing purposes under prevailing legislation applicable to the research facility where such experimentation occurs.

As discussed above, prior to the present invention MEMO was previously uncharacterised in terms of any function. Nevertheless in the light of the disclosure herein it can be seen that modulators of MEMO, such as antibodies which bind it and hence can inhibit its interactions have utility e.g. in methods of investigating or controlling late stage migration.

Thus further aspects of the invention relate to the production of antibodies able to bind MEMO specifically, these antibodies per se, and use of them in the methods disclosed herein.

MEMO antibodies are specific in the sense of being able to distinguish between the polypeptide it is able to bind and other polypeptides of the same species for which it has no or substantially no binding affinity (e.g. a binding affinity of at least about 1000× worse). Specific antibodies bind an epitope on the molecule which is either not present or is not accessible on other molecules.

Preferred antibodies according to the invention are isolated, in the sense of being free from contaminants such as antibodies able to bind other polypeptides and/or free of serum components. Monoclonal antibodies are preferred for some purposes, though polyclonal antibodies are within the scope of the present invention.

Antibodies may be obtained using techniques which are standard in the art. Methods of producing antibodies include immunising a mammal (e.g. mouse, rat, rabbit) with a polypeptide of the invention. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992).

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, and a bivalent fragment including two Fab fragments linked by a disulphide bridge at the hinge region. Single chain Fv fragments are also included.

Humanized antibodies in which CDRs from a non-human source are grafted onto human framework regions, typically with the alteration of some of the framework amino acid residues, to provide antibodies which are less immunogenic than the parent non-human antibodies, are also included within the present invention

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB-A-2188638 or EP-A-0239400. Cloning and expression of chimeric antibodies are described in EP-A-0120694 and EP-A-0125023.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

One favoured mode is by covalent linkage of each antibody with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

The mode of determining binding is not a feature of the present invention and those skilled in the art are able to choose a suitable mode according to their preference and general knowledge.

Antibodies according to the present invention may be used in screening for the presence of a polypeptide, for example in a test sample containing cells or cell lysate as discussed, and may be used in purifying and/or isolating a polypeptide according to the present invention, for instance following production of the polypeptide by expression from encoding nucleic acid therefor. Antibodies may modulate the activity of the polypeptide to which they bind and so, if that polypeptide has a deleterious effect in an individual, may be useful in a therapeutic context (which may include prophylaxis).

An antibody may be provided in a kit, which may include instructions for use of the antibody, e.g. in determining the presence of a particular substance in a test sample. One or more other reagents may be included, such as labelling molecules, buffer solutions, elutants and so on. Reagents may be provided within containers which protect them from the external environment, such as a sealed vial.

The present inventors have shown that Tyr residue 1201 and 1227 have a unique function in cell motility that cannot be substituted by other ErbB2 phosphorylation sites. Superficially, Y1201 and Y1227 appear to play similar roles in ErbB2 dependent motility since both mediate transcription dependent late stage motility. However, the present inventors have shown that MEMO is required downstream of phosphorylated Y1227 but not Y1201. Therefore, in another aspect the invention relates to other mediators of cell migration which are mediators of Y1201-induced migration and can be found in the light of the present disclosure. Preferably, these mediators are not mediators of Y1227-induced migration, such that inhibition of these mediators does not affect cell motility signalling from Y1227. More preferably, they are mediators of late stage migration events.

The present invention also relates to a method of providing a binding partner of MEMO, which may be suitable for use in one or more of the methods described above.

In one embodiment, the method comprises:
providing a MEMO polypeptide;
contacting the MEMO polypeptide with a MEMO binding partner;
determining whether the MEMO binding partner is able to bind to the MEMO polypeptide.

It is reiterated that the MEMO polypeptide may be a variant or fragment of MEMO, as defined above. It is preferred that the variant or fragment is a variant or fragment which retains at least one function of MEMO, preferably the ability to mediate migration events, more preferably migration events induced by tyrosine kinase receptors, more preferably by ErbB2, and most preferably by phosphorylation of Y1227 of ErbB2. Preferably the migration events are late stage and not early stage.

The binding partner of MEMO may be a binding partner which is upstream or downstream in the signalling pathway, wherein factors which are upstream in the pathway modulate (e.g. activate) MEMO and factors which are downstream in the pathway are modulated (e.g. activated) by MEMO.

For identifying a downstream binding partner, it is preferred that MEMO is provided in an activated form. “Activated” will be understood by those skilled in the art to mean in an appropriate environment (and including appropriate factors) to demonstrate activity e.g. present in a membrane).

In this embodiment, the non-activated form of MEMO may be used as a control to determine binding specificity. Examples of methods of providing activated MEMO have be described above.

In these binding assays, the association between MEMO and its natural binding partner may be direct or indirect. For example, there may be “adaptor” molecules which are required for the physical association to occur.

The method may be carried out in vitro. In one embodiment, the MEMO polypeptide may be immobilised on a solid support. Methods for doing this have been described. The immobilised polypeptide may then be contacted with a MEMO binding partner.

In one embodiment, the immobilised polypeptide may be contacted with a sample which contains multiple potential binding partners. Unbound material can be washed away, and bound material released for example by contacting it with a detergent such as SDS. The identity of protein bound to the immobilised polypeptide may then be assessed by any of the methods known to the skilled person, including SDS PAGE and mass spectrometry.

If the MEMO polypeptide is contacted with a sample comprising only one potential binding partner, then the methods for studying the interaction between the polypeptide and the binding partner may be substantially as described with reference to assay methods for inhibitors. For example, they may comprise labelling one of the polypeptide and its binding partner with a detectable label, and immobilising the other. The amount of binding can then be assessed by determining the amount of label bound to the solid support. In an alternative, fluorescence resonance energy transfer may be used, as previously described. If the test compound is in fact a binding partner then the fluorescence signal of the donor will be altered.

Assays for binding partners may also be carried out in vivo, e.g., by using a yeast two hybrid method as previously described.

MEMO polypeptides may be expressed as fusion proteins with an appropriate domain and candidate second polypeptides with which those of the invention might associate can be produced as fusion proteins with an appropriate corresponding domain. Alternatively libraries such as phage display libraries of such fusion proteins may be screened with a fusion polypeptide of the invention.

In various embodiments of this aspect, the methods of identifying binding partners of MEMO may be useful as methods of identifying specific mediators of migration events, more preferably a specific mediator of late stage over early stage events.

In such embodiments the methods of the invention may further comprise an additional, functional screening step of confirming that the MEMO binding partner is a specific mediator of cell motility or migration events, more preferably a specific mediator of late stage over early stage events.

A “mediator” of cell motility as used herein is a naturally occurring intermediate in a signalling pathway which positively regulates cell motility in response to a signal. Thus a mediator of late stage cell motility is a member of a pathway which positively regulates late stage cell motility in response to a signal. A mediator of late stage but not early stage cell migration events is a member of a pathway which positively regulates late stage events, and of which the activity in that late stage pathway can be inhibited without significantly inhibiting early events.

Thus in one embodiment, the methods described above may include the subsequent step of:
providing a cell in which the activity of the MEMO binding partner is modulated;
detecting whether early stage migration events are affected by said modulation; and\or
detecting whether late stage migration events are affected by said modulation.

Preferably, the modulation is inhibition, and these methods are analogous to those which were used to identify specific MEMO-based inhibitors of late-stage cell migration events.

In one embodiment, the cell used may be motile in the absence of exogenous stimuli. Here, the migration-inducing signal may be provided constitutively by the cell. An example of such a cell is a MDA-MB-231 cell. In another embodiment, the signal is provided exogenously, e.g., by contacting the cell with a ligand for a tyrosine kinase receptor, such as a ligand for the FGF2, EGF receptors of for ErbB2.

In a preferred embodiment, the method comprises determining whether the MEMO binding partner is a mediator of migration events induced by ErbB2 activation. In this embodiment, the motility-inducing signal is provided by activation of the ErbB2 receptor, for example by constitutive activation of ErbB2 in the cell or by contacting the receptor with a ligand of ErbB2, such as heregulin.

The cell in which activity of the MEMO binding partner is modulated may be provided by targeted mutagenesis, by downregulation of translation (using for example siRNA or antisense RNA), or by contacting the cell with a specific inhibitor of the MEMO binding partner's activity.

The most preferred target of the present application is the late effect pathway stimulated by Y1201 or Y1227 of ErbB2, as over expression of ErbB2 has been implicated in cancer metastasis. Accordingly, in a further embodiment, the assay method may comprise, subsequent to identifying a MEMO binding partner, determining whether the binding partner is a mediator of migration events induced by Y1201 or Y1227 phosphorylation, e.g., as described in the examples.

The present inventors have also observed that mediators which are required for early stage cell migration are necessary for efficient migration upon activation of either Y1201 or Y1227 (i.e., they seem to be required generally for migration). In contrast, it appears that the “late stage” mediators, or at least those which act immediately downstream of ErbB2, are required for signal transduction from either Y1201 or Y1227 but not both. This again indicates that the signalling pathways mediated downstream of ErbB2 Y1201 and Y1227 may have a more specialised role than the pathways previously implicated in migration.

This observation provides the basis for an alternative methodology for establishing whether a given mediator is a specific mediator of late-stage migration events, which may be applicable to any of the methods discussed herein in which it is desired or required to establish this.

Thus, in one embodiment, the method for determining whether the MEMO binding partner is a specific mediator of late-stage migration events may comprise:

determining whether the MEMO binding partner is required for signal transduction from ErbB2 residues Y1201 or Y1227;
selecting the mediator if it is required for signal transduction from Y1227 but not Y1201.

This may be done in one embodiment by:
i) providing a first cell in which ErB2-induced cell migration signalling is mediated by Y1227 and not Y1201;
ii) providing a second cell in which ErbB2-induced cell migration signalling is mediated by Y1201 and not Y1227;
iii) inhibiting the activity of the MEMO binding partner of interest in said cells;
iv) selecting the MEMO binding partner if said inhibition inhibits cell migration in the cells of step i) but not step ii).

As above, a cell in which ErbB2-induced cell migration signalling is induced by only one of Y1201 or Y1227 can be achieved by providing a cell which expresses a form of ErbB2 in which the tyrosine at the other autophosphorylation site has been replaced by a residue which is not susceptible to phosphorylation.

The present inventors have further found that the late stage events of cell migration appear to be dependent on de novo RNA and protein synthesis. In particular, phosphorylation of Neu/ErbB2 on Tyr1201 or Tyr1227 activated those stages of cell migration that are transcription/translation dependent. This has not been previously observed. This novel observation provides a method of identifying further mediators of late-stage cell migration, which comprises comparing mRNA and/or protein expression in:

i) a cell which has received a migration-inducing signal and which has not been contacted with an inhibitor of late stage migration events; with
ii) a cell which has not received a migration inducing signal or which has received a migration-inducing signal and which has been contacted with an inhibitor of late-stage migration events.

Preferably, the inhibitor is of late stage preferentially over early stage cell migration events. mRNA transcripts and proteins which are expressed at different levels in the two cell types may be identified.

The migration-inducing signal may be provided as previously described, and is preferably a ligand for ErbB2.

In a preferred embodiment, the inhibitor of late stage migration events is an inhibitor of MEMO activity, which may for example be provided using one of the above-described methods.

Protein expression in the two cells may be assessed using any of the proteomics techniques known in the art (see for example Resing K A, Ann N Y Acad Sci 2002 October; 971: 608-14, or MacBeath G. Nat Genet 2002 December; 32, Suppl: 526-32.)

mRNA expression in the cells may be assessed by any of the microarray techniques known in the art (see for example the Affymetrix GeneChip Expression Analysis Technical Manual, Brown P. O and Bottstein D., Nat Genet 1999 January; 21 Suppl:33-37, Lipshutz et al., Nat Genet 1999 January; 21 Suppl:20-24, Harkin D P.; Oncologist 2000; 5(6):501-7, Heller M J., Annu Rev Biomed Eng 2002; 4:129-53). For data analysis, see for example Slonim D K., Nat Genet 2002 December; 32 Suppl:502-8 and Butte A., Nat Rev Drug Discov 2002 December; 1(12):951-60.

Where a proteinaceous binding partner is identified using any of the methods described above this may, if desired, be further purified as an active protein from a mixture using techniques well known to those skilled in the art, and further isolation of the mediator, in the light of the present disclosure, will present no burden to those of ordinary skill in the art. Typical protocols are set out “Protein Purification—principles and practice” Pub. Springer-Verlag, New York Inc (1982), and by Harris & Angal (1989) “Protein purification methods—a practical approach” Pub. O.U.P. UK, or references therein.

A typical protocol for obtaining the mediator may include:

(i) preparing cell free fluid comprising the mediator, followed by one, preferably two or more of the following steps (in any order):
(ii) gel filtration of the supernatant;
(iii) ion exchange chromatography (anion or cation exchange),
(iv) hydrophobic interaction chromatography.

At each stage purification and yield can be assessed e.g. using the binding assays discussed above. Further characterisation during or following the procedure may involve MALDI-TOF mass spectrometry (e.g. using the Voyager DE-PRO system) and/or SDS-PAGE.

Proteins associated with active fractions may be fully or partially sequenced, optionally following SDS-PAGE. The sequence information may be compared with that on databases to identify sequences which may have this activity.

Suitable inhibitors (e.g. which modulate the late stage pathways discussed above e.g. via modulation of the activity of mediators within those pathways, such as those identified in the functional or other assays discussed above) may be incorporated into medicaments e.g. after further testing for toxicity. These include siRNAs and related vectors as discussed above.

Also, as discussed, in another embodiment the method comprises the use of MEMO (including fragments or variants thereof) to treat conditions which benefit from enhanced cell migration (e.g., wound healing). Accordingly, it will be understood that the discussion of inhibitors below may also apply to MEMO.

Thus the relevant methods may include the further step of formulating a selected inhibitor as a medicament for a disease e.g. in which it is desired to control cell motility e.g. the treatment of tumors, including breast, ovary, lung, prostate or gastric carcinomas. The medicament may also be used for the treatment of angiogenesis, which involves the migration of endothelial cells. Such inhibitors and medicaments for use in the treatment of these diseases, and methods of treatment comprising their use form further aspects of the invention.

The term treatment as used herein is intended to include prophylaxis and prevention as well as alleviation of the condition or symptoms thereof.

The compositions may include, in addition to the above constituents, pharmaceutically-acceptable excipients, preserving agents, solubilizers, viscosity-increasing substances, stabilising agents, wetting agents, emulsifying agents, sweetening agents, colouring agents, flavouring agents, salts for varying the osmotic pressure, buffers, or coating agents. Such materials should be non-toxic and should not interfere with the efficacy of the active ingredient. The precise nature of the carrier or other material may depend on the route of administration. Examples of techniques and protocols can be found in “Remington's Pharmaceutical Sciences”, 16^{th} edition, Osol, A. (ed.), 1980.

Where the composition is formulated into a pharmaceutical composition, the administration thereof can be effected parentally such as orally, nasally (e.g. in the form of nasal sprays) or rectally (e.g. in the form of suppositories). However, the administration can also be effected parentally such as intramuscularly, intravenously, cutaneously, subcutaneously, or intraperitoneally (e.g. in the form of injection solutions).

Thus, for example, where the pharmaceutical composition is in the form of a tablet, it may include a solid carrier such as gelatine or an adjuvant. For the manufacture of tablets, coated tablets, dragees and hard gelatine capsules, the active compounds and their pharmaceutically-acceptable acid addition salts can be processed with pharmaceutically inert, inorganic or organic excipients. Lactose, maize, starch or derivatives thereof, talc, stearic acid or its salts etc. can be used, for example, as such excipients for tablets, dragees and hard gelatine capsules. Suitable excipients for soft gelatine capsules are, for example, vegetable oils, waxes, fats, semi-solid and liquid polyols etc. Where the composition is in the form of a liquid pharmaceutical formulation, it will generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may also be included. Other suitable excipients for the manufacture of solutions and syrups are, for example, water, polyols, saccharose, invert sugar, glucose, trihalose, etc. Suitable excipients for injection solutions are, for example, water, alcohols, polyols, glycerol, vegetable oils, etc. For intravenous, cutaneous or subcutaneous injection, or intracatheter infusion into the brain, the active ingredient will be in the form of a parenterally-acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers and/or other additives may be included, as required.

The disclosure of any cross-reference made herein, inasmuch as it may be required by one skilled in the art to supplement the present disclosure, is hereby specifically incorporated herein.

The invention will now be further described with reference to the following non-limiting Figures and Examples. Other embodiments of the invention will occur to those skilled in the art in the light of these.

### FIGURES

FIG. 1a: shows the migration response of the T47D breast carcinoma cell line and of T47D-5R carcinoma cell lines to heregulin signalling.

FIG. 1b: is a schematic representative of the Neu, NYPD, YA, YB, YC, YD and YE proteins

FIG. 1c: shows the migration response of Neu-, NYPD-, YA-, YB, YC-, YD- and YE-expressing T47D-5R cell lines to heregulin signalling.

FIGS. 2a, 2b, 2c and 2d: shows the heregulin-induced migration response of YC- and YD-expressing cells transfected with YC, phosphorylated YC (pYC), YD or phosphorylated YD (pYD) peptides.

FIG. 3a: shows the morphological response of heregulin-treated T47D and NYPD cells over time.

FIG. 3b: shows the kinetics of Rac activation in heregulin treated T47D and NYPD cells over time.

FIG. 3c: shows the extent of lamellipodia formation in heregulin-treated T47D cells over time, quantified using the method described below.

FIG. 3d: shows the extent of lamellipodia formation in heregulin treated Neu- and NYPD-expressing T47D-5R cell lines.

FIG. 4a: shows the heregulin-induced migration response of Neu- and NYPD-expressing T47D-5R cell lines in the presence and absence of cyclohexamide (CHX).

FIG. 4b: shows the heregulin-induced migration of NYPD-, YC-, YD- and YE-expressing T47D-5R cell lines over time in the presence or absence of CHX.

FIG. 5a: shows the heregulin-induced migration of Neu-expressing T47D-5R cells in the presence of MEK, PI3K, p38 or Src inhibitors.

FIG. 5b: shows the heregulin-induced activation of the MAPK, PI3K and p38MAPK pathways in Neu-, NYPD-, YA-, YB-, YC-, YD- and YE-expressing T47D-5R cell lines over time. The activation of these pathways was assessed by western blotting of cell extracts, followed by probing of the membranes with P-MAPK, P-PKB and P-p38 antibodies.

FIG. 5c: shows heregulin-induced migrations of NYPD-expressing T47D-5R cell lines cells in the presence of MEK, PI3K, p38 or Src inhibitors.

FIG. 5d: shows the effect of MEK and PI3K inhibitors on HRG-induced lamillipodia formation in Neu- and NYPD-expressing T47D-5R cell lines.

FIG. 6a: shows binding of Shc, CrkIII, PLCγ and MEMO to YC, pYC, YD and pYD peptides. This was analyzed by pull-down, followed by Western blotting with the respective antibodies. Memo/CGI-27 was pulled down from cells expressing GFP-Memo fusion protein and probed with an anti-GFP antibody. Whole cell extracts (W) were also loaded on the gel.

FIG. 6b: shows Memo cellular localization in control cells and after 5 min HRG stimulation. This was visualized in Myc-Memo expressing SKBr3 cells, using an anti-Myc antibody. Nuclei were stained with DAPI.

FIG. 6c: shows HRG-dependent migration of YC and YD cells, tested after Shc siRNA transfection. Protein extracts were collected 3 and 4 days (3d and 4d) after transfection. The effect of Shc siRNA on Shc expression was verified by Western blotting using a Shc specific antibody (insert).

FIG. 6d: shows the migration of YC- and YD-expressing T47D-5R cells in response to HRG, in the absence or presence of a PLC inhibitor.

FIG. 6e: shows HRG-dependent lamellipodia outgrowth in Neu and NYPD cells, in the presence of Shc siRNA or a PLC inhibitor.

FIG. 7a: shows HRG-dependent migration of YD-expressing T47D-5R cells treated with control (LacZ) or Memo siRNA. RNA was collected 3d and 4d after transfection and Memo mRNA was measured by quantitative PCR (insert).

FIG. 7b: shows the effect of Memo siRNA on HRG-induced migration of Neu-, NYPD-, YC- and YD-expressing T47D-5R cells.

FIG. 7c: shows HRG-induced lamellipodia formation in YD-expressing T47D-5R cells, in the presence of control (LacZ) or Memo siRNA.

FIG. 7d: shows the effect of Memo siRNA on migration of YD-expressing T47D-5R cells treated with cycloheximide (CHX).

FIG. 7e: shows HRG-dependent migration of T47D, SKBr3 and MDA-MB-231 cells after Memo siRNA transfection.

FIG. 7f: shows the effect of Memo siRNA on migration of T47D cells in response to HRG, FGF2, insulin and EGF.

### EXAMPLES

### Materials and Methods

### Plasmid Constructs, Cell Culture, Cell Transfection:

T47D and SKBr3 breast carcinoma cells were cultured in Dulbecco's modified Eagle's medium supplemented with 10% fetal calf serum (GIBCO Invitrogen A G, Basel, Switzerland). T47D-5R cells were obtained by infection of T47D cells with a pBabe-based retrovirus expressing the scFv-5R cDNA as previously described²⁴. The infected cells were selected in 1 mg/ml G418 (GIBCO) and clones were generated and tested for the absence of surface ErbB2 by FACS. Cells were then transfected with plasmids encoding Neu or Neu add back mutants¹³ using FuGene (Roche Diagnostics Corporation, Indianapolis, Ind., USA) and selected in 1 μg/ml puromycin (Sigma, St. Louis, Mich., USA). In order to obtain cells expressing similar amounts of Neu receptor, cells were sorted after surface staining with a Neu specific antibody (oncogene, Darmstadt, Germany).

Memo cDNA, obtained by RT-PCR using mRNA of T47D cells as a template, was first cloned into pGEM-T Easy (Promega Corporation, Madison, Wis., USA), then subcloned into pcDNA3-derivatives containing either the GFP or the Myc epitope to generate the N-terminally tagged fusion proteins, GFP-Memo and Myc-Memo. Constructs were verified by sequence analysis and transfected into SKBr3 cells using FuGene.

### Migration Assay:

Cell migration was tested using 8 μm-pore polycarbonate membrane Transwell chambers (Corning Costar Products, Acton, Mass., USA) as described previously³. In brief, the bottom side of the membrane was coated with 25 μg/ml rat tail collagen I (Roche). Serum starved cells were plated in the top Transwell chamber. Medium with or without 1 nM HRG-β1 (R&D systems, Inc., Minneapolis, Minn., USA) was added to the bottom chamber and cells were allowed to migrate for 24 hours. Non-migrated cells were scraped off the top of the membrane. Migrated cells were fixed in 4% formaldehyde and stained in 0.1% crystal violet. Cells were counted under a microscope in ten high power fields. Migration was expressed as cell number per mm². In some instances, cells were pre-incubated for 60 minutes with the U0126 MEK inhibitor (50 μM; Promega), the LY294002 PI3K inhibitor (50 μM; Calbiochem-Novabiochem Corporation, San Diego, Calif., USA), the SB203580 p38 MAPK inhibitor (20 μM; Calbiochem), a Src inhibitor (2.5 μM), the U-73122 PLC inhibitor (2 μM; Calbiochem), cycloheximide (10 μg/ml; Sigma) or 5,6-dichloro-1-β-D-ribofuranosylbenzimidazole (20 μg/ml; Fluka Chemie GmbH, Buchs, Switzerland). Cells were then allowed to migrate for 8 hours before counting.

### Western Blot:

Cells were lysed in NP-40 lysis buffer (50 mM Hepes pH 7.4, 150 mM NaCl, 25 mM β-glycerol phosphate, 25 mM NaF, 5 mM EGTA, 1 mM EDTA, 1% NP-40, 10 μg/ml leupeptin, 10 μg/ml aprotinin, 10 μg/ml sodium vanadate and 100 μM phenylmethylsulfonyl fluoride) for 5 minutes on ice. Lysates were centrifuged at 14,000 g for 20 minutes. Proteins were blotted on polyvinylidene difluoride membrane (Millipore GmbH, Vienna, Austria) and membranes were blocked with 10% horse serum (GIBCO) in 50 mM Tris pH 7.5, 150 mM NaCl. Filters were incubated with specific antibodies against P-p44/42, P-Akt/PKB and P-p38MAPK (from New England Biolabs, Beverly, Mass., USA) for 2 hours. Proteins were visualized with peroxidase-coupled secondary antibodies using the enhanced chemiluminescence detection system (Amersham Pharmacia Biotech, Dübendorf, Germany).

### Rac Activity Assay:

Active Rac was detected using a glutathione-S-transferase (GST)-PAK-CD (PAK-CRIB domain) fusion protein as described previously³⁸. Shortly, cells were plated on collagen-coated dishes and lysed on ice with lysis buffer (50 mM Tris-HCl pH 7.4, 2 mM MgCl₂, 1% NP-40, 10% glycerol, 100 mM NaCl, 10 μg/ml leupeptin, 10 μg/ml aprotinin, 10 μg/ml sodium vanadate and 100 μM phenylmethylsulfonyl fluoride). Lysates were clarified by centrifugation at 14,000 g for 5 minutes. Aliquots from the clarified lysates were taken in order to analyze total amount of Rac1. Clarified lysates were incubated with bacterially produced GST-PAK-CD fusion protein bound to glutathione-coupled Sepharose beads. Proteins bound to the fusion protein were analyzed by Western blotting using an anti-Rac1 antibody (Upstate biotechnology, Lake Placid, N.Y., USA).

### Immunofluorescence, Actin Staining:

Cells were grown on glass coverslips (Falcon, Le Pont De Claix, France) coated with 25 μg/ml rat tail collagen I, serum starved overnight and stimulated with 1 nM HRG-β1 for different times. Cells were fixed in 4% formaldehyde in phosphate buffer saline (PBS) for 20 minutes, permeabilized in 0.2% Triton X-100 for 10 minutes, blocked with 1% bovine serum albumin in PBS for 20 minutes before addition of anti-Myc antibody (Santa Cruz) and Alexa-Fluor 568 goat anti-mouse IgG (Molecular Probes, Leiden, The Netherlands). DNA was counterstained with 0.25 mg/ml Hoechst No. 33342 (Sigma). Actin was stained for 45 minutes with 2 U/ml TRITC-labeled phalloidin (Molecular Probes). Images were recorded with an Axioskop Zeiss microscope coupled to a Sony 3CCD camera or an Olympus IX70 microscope linked to the DeltaVision workstation (Applied Precision, Issaquah, Wa)

### Visualisation and Quantification of Lamellipodia:

F-actin was visualised with TRITC-labelled phalloidin.

Proteins localized n lamellipodia were specifically purified using the method described by Cho et al.³⁹. Briefly, cells were plated on 3 μm porous polycarbonate membrane Transwell chamber (Costar) coated on the bottom side with rat tail collagen I. The lower chamber contained medium with or without 1 nM HRG-β1. Cells were allowed to extend pseudopodia through the pores for different times. Cell bodies remaining on the upper surface were removed and the pseudopodia extending to the lower surface were lysed (100 mM Tris pH 7.4, 5 mM EDTA, 150 mM NaCl, 1% sodium dodecyl sulfate, 10 μg/ml leupeptin, 10 μg/ml aprotinin, 10 μg/ml sodium vanadate and 100 μM phenylmethylsulfonyl fluoride). Protein concentration was measured using Bio-Rad Dc protein assay (Bio-Rad Laboratories, Hercules, Calif., USA), and the results expressed in mg/ml. This approach can be used to quantify lamellipodia.

### SiRNA and Peptide Transfection:

Cells were transfected with siRNA using Oligofectamine (GIBCO) according to the manufacturer's instructions. The following 21-mer oligoribonucleotide pairs (obtained from Xeragon Inc., Huntsville, Ala., USA) were used: for Shc (accession number HSU7377) nucleotide 236 to 25640, for Memo (CGI-27; accession number AF132961) nucleotide 460 to 480 and for control LacZ (accession number M55068) nucleotide 4277 to 4297. Cells were plated for migration assay 3 days after siRNA transfection and allowed to migrate for 24 hours. Cell lysates were also prepared 3 and 4 days after transfection and analyzed by Western blotting using a specific anti-Shc antibody (BD Transduction laboratories, Heidelberg, Germany). For Memo, mRNA was extracted using RNeasy Mini (Qiagen, Cologne, Germany) and quantitative radioactive PCR⁴¹ was performed using Memo specific primers (forward from nucleotide 90 to 111 and reverse from nucleotide 235 to 256).

Peptides were delivered into cells using Chariot protein transfection reagent (Active Motif, Rixensart, Belgium) according to the instruction manual. 16 aa-peptides spanning Tyr residue 1201 of Neu (YC peptide) and Tyr residue 1227 of Neu (YD peptide) were obtained from Neosystem (Strasbourg, France) in phosphorylated and non phosphorylated forms. Cells were plated for migration 30 minutes after peptide transfection and the assay was finished after 22 hours.

### Pull Down Assay and Mass Spectrometry:

Phosphorylated and non-phosphorylated YC and YD peptides were coupled under anhydrous conditions to Affi-gel 10 agarose beads (Bio-Rad). Coupled beads were incubated with 0.5 mg (for Western blotting) or 12 mg (for mass spectrometry) T47D cell lysates. Proteins bound to the peptides were subjected to SDS-PAGE. For mass spectrometry the gels were stained with Coomassie Brilliant Blue R-250. Each lane of the gels was sliced and analyzed by LC-MSMS (LCQ Deca XP, Thermo Finnigan) and proteins identified by Turbo Sequest. Proteins identified by more than two peptides and binding specifically to the phosphorylated form of the peptides were selected for further analysis. Binding was confirmed by Western blotting using antibodies against CrkII and PLCγ from Santa Cruz and Shc from BD Transduction laboratories. For Memo, pull-downs were performed on extracts from SKBr3 cells expressing a GFP-Memo fusion protein and analyzed by Western blotting using an anti-GFP antibody (Santa Cruz).

In some experiments, Shc was immunodepleted from SKBr3 cells expressing Myc-Memo fusion protein or from reticulocyte lysates expressing in vitro translated Myc-Memo using the anti-Shc antibody, before performing the pull-down.

### Results

### Role of Specific ErbB2 Tyrosine Residues in Heregulin-Induced Migration

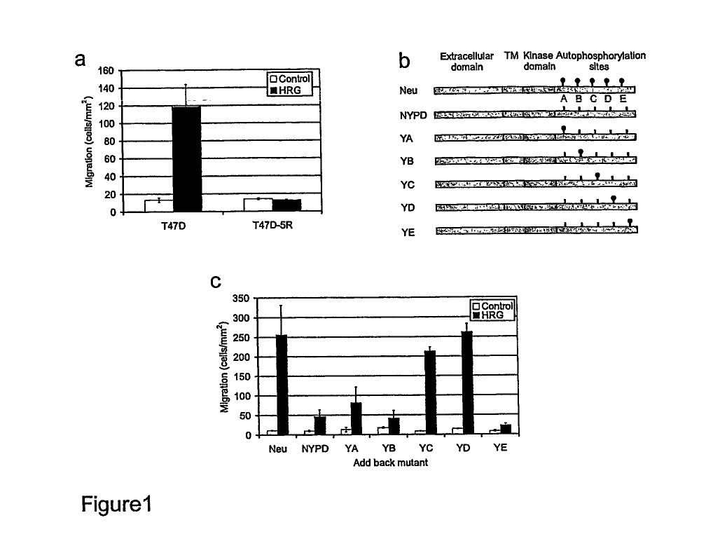

It has previously been shown that the T47D breast carcinoma cell line, which expresses moderate levels of the four ErbB receptors, is dependent upon ErbB2 activity for migration in response to EGF-related ligands⁸. To explore this in more detail, the inventors have investigated the role of individual ErbB2 autophosphorylation sites in migration. Initially, ErbB2 was functionally inactivated in T47D cells by expressing a single chain antibody (scFv-5R) that traps human ErbB2 in the endoplasmic reticulum²⁴, thus inhibiting its transfer to the plasma membrane, as confirmed by the absence of ErbB2 surface staining and preventing ligand-induced ErbB2 activation. Migration of wild type and scFv-5R-expressing cells (T47D-5R) in response to heregulin β1 (HRG) was measured in Boyden-like chambers. HRG binding to ErbB3 and ErbB4 leads to the formation of active ErbB2-containing heterodimers. HRG stimulated migration of T47D cells very efficiently, while T47D-5R cells were unable to migrate above basal levels (FIG. 1a), confirming the essential role of ErbB2 in EGF-related peptide induced migration.

T47D-5R cells were used as recipients of vectors expressing WT Neu, the rat homologue of ErbB2, or mutant Neu with a Phe residue substituted in each of the five autophosphorylation sites (called NYPD for Neu Tyr phosphorylation deficient) or Neu add-back mutants expressing only one of the five autophosphorylation sites, called YA, YB, YC, YD and YE, corresponding to Tyr1028, Tyr1144, Tyr1201, Tyr1227 and Tyr1253, respectively (nomenclature according to Dankort et al.¹³) (FIG. 1b). Cells expressing similar levels of Neu were selected and their migration in response to HRG was evaluated. Neu efficiently replaced ErbB2 in T47D-5R cells, as demonstrated by their restored migratory response to HRG (FIG. 1c). In contrast, NYPD-, YA-, YB- and YE-expressing cells showed strongly reduced migration in response to HRG. It should be mentioned that each Neu mutant, despite lacking autophosphorylation sites, can interact with and transphosphorylate the other HRG-bound ErbB receptors, which likely explains the ability of these cells to migrate above the basal level observed in the T47D-5R cells (FIG. 1a).

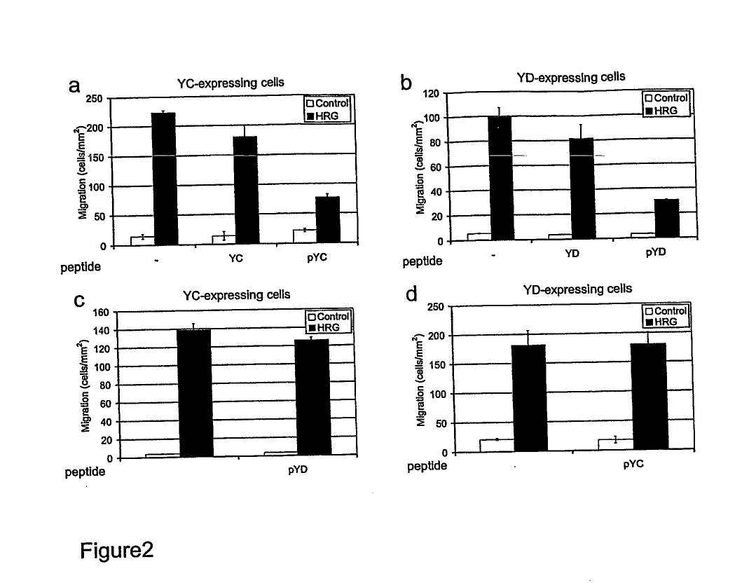

Migration of YC- and YD-expressing cells was equivalent to that of Neu-expressing cells (FIG. 1c), indicating that these two tyrosine residues couple to signaling pathways required for efficient cell migration. To verify their proposed role, tyrosine phosphorylated or non-phosphorylated peptides, corresponding to the region of Neu including the YC or YD residues, were used to compete for binding of signaling molecules to Neu. Transfection of YC-expressing cells with a phospho-YC peptide prevented HRG-induced migration (FIG. 2a), while the non-phosphorylated peptide did not interfere significantly with migration. Similarly, only the phospho-YD peptide efficiently inhibited migration of YD-expressing cells (FIG. 2b). Moreover, the phospho-YD peptide did not inhibit migration of YC-expressing cells (FIG. 2c) and conversely phospho-YC peptide did not interfere with migration of YD-expressing cells (FIG. 2d). These results not only confirm the requirement for phosphorylation of YC or YD tyrosine residues for cell migration, but also show that these two tyrosines impinge on distinct signaling molecules.

### HRG-Induces Morphogenetic Changes in T47D and NYPD Cells

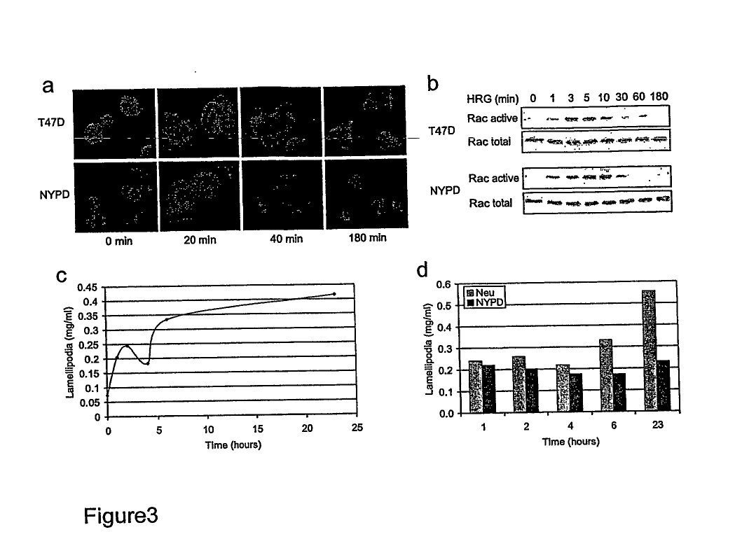

Cell motility can be viewed as a series of morphogenetic events based on remodeling of the actin cytoskeleton. Thus, the inventors analyzed HRG-induced changes in cell morphology and cytoskeleton organization in migratory and non-migratory cells. HRG-treated T47D cells rapidly spread and formed membrane ruffles. Initially, cells extended lamellipodia in all directions, before showing a more polarized organization, paralleling the formation of actin stress fibers (FIG. 3a, upper panel). NYPD-cells, while greatly impaired in migration (FIG. 1c), displayed a normal morphogenetic response, extending and organizing lamellipodia after HRG treatment (FIG. 3a, lower panel). Lamellipodia formation is dependent on the activation of Rac, a member of the Rho GTPase family²⁶. The kinetics of HRG-induced Rac activation was similar in T47D- and NYPD-cells; in both cell lines activity was transient, peaking 5 min after HRG addition (FIG. 3b). These results are in accordance with the morphological results, and provide further evidence that T47D- and NYPD-cells undergo comparable cytoskeletal rearrangements shortly after HRG treatment.

Cytoskeleton remodeling is widely used as a read-out for cell motility. The fact that HRG-triggered actin reorganization in T47D and NYPD cells was essentially identical, was in apparent contradiction with the difference they actually demonstrate in migration. To minimize variations due to assay conditions, the inventors analyzed lamellipodia formation in the same dual-chamber setting used to measure cell migration. T47D cells show a rapid increase in lamellipodia, apparent within an hour of HRG treatment, followed by a plateau and a second slower increase after 6 hrs (FIG. 3c). Lamellipodia formation during the early time points (up to 4 hours) was similar in Neu cells and in NYPD cells, but was strikingly different at later times (FIG. 3d). These results confirm that the early morphological and molecular changes occurring in response to HRG are similar in migratory and non-migratory cells. Furthermore, they suggest that migration of NYPD cells is affected at a later stage.

### HRG-Induced Migration Requires De Novo RNA and Protein Synthesis

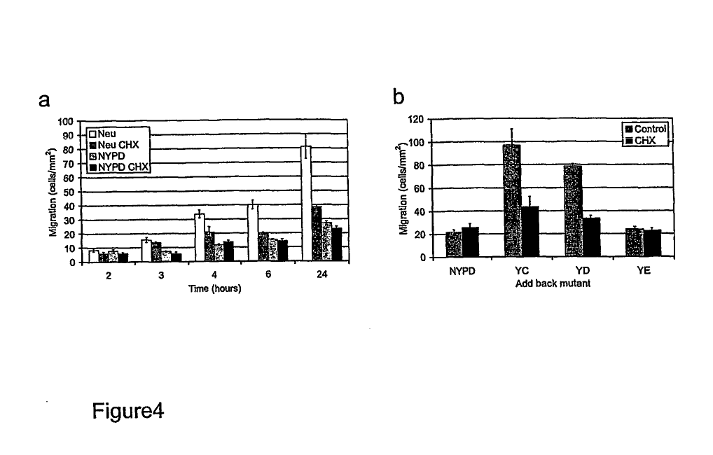

In the next experiment the inventors analyzed HRG-induced migration of Neu- and NYPD-cells over a 24 hr time course. Neu cells migrated within 2 hrs of HRG treatment and the number of cells increased steadily up to 24 hrs (FIG. 4a). After 2 hrs, migration of NYPD cells was equivalent to that of Neu cells. Thereafter, however, their migration increased at a very low rate, supporting the hypothesis that the migratory ability of NYPD cells is blocked at later time points. The inventors next explored the possibility that efficient, long-term migration required de novo RNA or protein synthesis, using respectively, 5,6-Dichloro-1-β-D-ribofuranosylbenzimidazole (DRB), an inhibitor of RNA polymerase II or cycloheximide. Starting 4 hrs after HRG treatment, migration of Neu cells was strongly blocked in the presence of cycloheximide (FIG. 4a) and DRB. Similarly, migration of YC- and YD-cells was sensitive to both inhibitors (FIG. 4b). In contrast, the low migration level of NYPD- and YE cells was not affected by the same treatments (FIGS. 4a, 4b). Cycloheximide reduced the migration rate of Neu cells to that of NYPD cells (FIG. 4a). This suggests that following HRG stimulation, post-translational events, e.g. phosphorylation, trigger moderate levels of migration, while efficient, long-term migration, the response that is lacking in NYPD cells, is mainly dependent on de novo RNA and protein synthesis.

### Signalling Pathways Involved in ErbB2-Dependent Migration

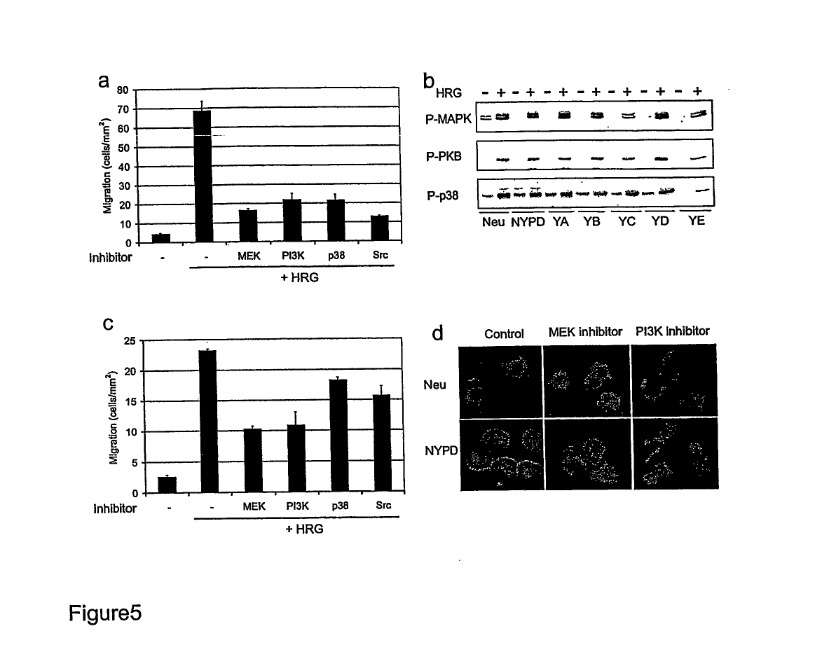

The present data shows that Neu/ErbB2, more specifically, the phosphorylated YC and YD residues, are crucial mediators of efficient, HRG-induced cell migration. Pathways implicated directly, or indirectly, in ErbB2-induced cytoskeleton remodeling and/or cell motility have previously been identified. These include the Ras/MAPK, PI3K, p38MAPK and Src kinase-dependent pathwayS²⁰⁻²³. Using selective kinase inhibitors on Neu cells, the inventors determined that blocking each of these pathways led to a strong inhibition of HRG-induced migration (FIG. 5a). While activation of these pathways is required for migration, it is, however, not sufficient. Indeed, stimulation of the MAPK, PI3K, p38MAPK (FIG. 5b) and Src pathways did not correlate with migration, since HRG activated each pathway as efficiently in, e.g., NYPD- or YA-cells, as in Neu cells (FIG. 5b). As mentioned previously, Neu mutants can transphosphorylate the other ErbB receptors (ErbB3 or ErbB4), which likely explains activation of the examined pathways.

The kinase inhibitors also had a negative effect upon the low level of HRG-induced migration observed for NYPD cells, with blockade of the MAPK and PI3K pathways having the strongest effect (FIG. 5c). Moreover, both inhibitors also prevented Neu and NYPD cells from forming lamellipodia in response to HRG (FIG. 5d). Thus, activation of the MAPK and PI3K pathways is essential for early stages of migration. In contrast, the inventors propose that phospho-YC or -YD provide links to novel signalling pathway(s) that promote efficient long-term migration.

### Identification of Signalling Molecules Binding to the YC and YD Residues

To search for novel proteins that might link phospho-YC and YD to signaling pathways mediating ErbB2-dependent migration, tyrosine-phosphorylated peptides, corresponding to the regions of Neu including the YC or YD residues, were coupled to agarose beads and employed as affinity reagents. The corresponding non-phosphorylated peptides served as controls. The inventors performed a large-scale systematic identification of proteins from T47D cell extracts that bound specifically to the phosphor but not to the non-phosphorylated peptides, by high-pressure liquid chromatography, tandem mass spectrometry (LC-MSMS).

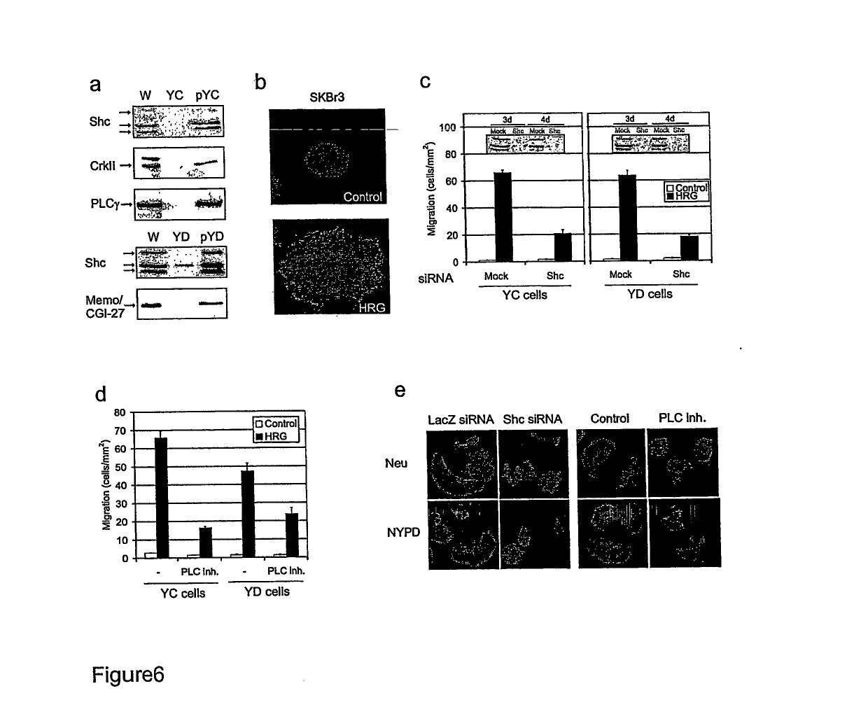

A number of proteins, some of which have been reported to bind ErbB2/Neu, others being novel interactors, were identified. Previous studies have shown that the adaptor molecules Shc and Crk II associated with the phospho-YD and -YC residues, respectively^{13,27}. The inventors also identified these two proteins in the LC-MSMS screen, finding in addition that Shc bound both phosphorylated peptides. Furthermore, not only CrkII, but also the CrkI splice variant and the Crk-like protein bound the phospho-YC peptide. Phospholipase Cγ (PLCγ) which has not previously been reported to interact with either of these Tyr residues, was found to associate with the phospho-YC peptide. Finally, a hypothetical protein, CGI-27 or c21 or f19-like protein, associated specifically with the phospho-YD peptide. CGI-27 was identified in three independent experiments from a total of six different peptides covering 38% of the sequence (113 out of 297 amino-acids). The binding specificity of each protein was confirmed in independent experiments using the phospho- and non-phosphorylated peptides as affinity reagents, followed by Western analysis (FIG. 6a). For CGI-27, for which no specific antibody is available, specific binding of a GFP-tagged version of the protein to the phospho-YD peptide is shown (FIG. 6a)-.

Analysis of CGI-27 sequence shows that it does not contain SH2 or PTB phosphotyrosine-binding domains. The fact that Shc also interacted with the phospho-YD site raised the possibility that Shc mediates the binding of CGI-27 to phospho-YD. In immunoprecipitation experiments, ectopically-expressed CGI-27 was found to interact with Shc in SKBr3 cells and both endogenous CGI-27 and ErbB2 were detected in Shc immunoprecipitates (results not shown). Intriguingly, blocking ErbB2 activity with PKI166 lowered the receptor level in Shc immunoprecipitates, but did not influence CGI-27's ability to associate with Shc, suggesting a constitutive association of the two proteins. To explore this further, Myc-CGI-27 was expressed in vitro in reticulocyte lysates and tested for binding. Immunodepletion of endogenous Shc from the lysates led to strongly decreased binding of Myc-CGI-27 to phospho-YD.

Interestingly, at the cellular level, CGI-27 was rapidly recruited and accumulated in discrete areas of the plasma membrane and the cytoplasm, upon HRG treatment (FIG. 6b).

### Role of PhosphoYC/PhosphoYD-Binding Proteins in Cell Migration

The identified proteins were next tested for their role in ErbB2-dependent cell migration. The function of Shc and Crk in HRG-induced migration was tested using small interfering (si) RNAs to block their expression. Specific siRNA transfection of YC or YD cells led to a strong decrease in the level of Shc (and Crk) relative to mock-transfected cells (FIG. 6c, inserts). In contrast to control cells, migration of YC and YD cells with knocked-down Shc (FIG. 6c) or Crk was considerably decreased. Furthermore, using a chemical inhibitor, the inventors found that phospholipase C activity was also necessary for HRG-induced migration of both YC- and YD-expressing cells (FIG. 6d). In the present experiments, however, PLCγ and Crk only bound phospho-YC, suggesting that the two are also mediators for other proteins, such as integrins, which are required for T47D cell migration⁸. Finally, siRNA-mediated knock-down of Shc (or CrkII) levels, or inhibition of PLC prevented Neu- and NYPD-cells from forming lamellipodia in response to HRG (FIG. 6e). Taken together, the results show that Shc, Crk and active PLCγ are required for HRG-induced cell migration. With respect to the migratory phenotype of T47D cells, however, none of these molecules has the characteristics of the protein the inventors are seeking; namely, one that binds to only one of the phospho-peptides (FIG. 2) and is involved in late stages of cell migration, but not in lamellipodia formation (FIGS. 3 and 4).

### CGI-27 is a Mediator of ErbB2-Dependent Motility

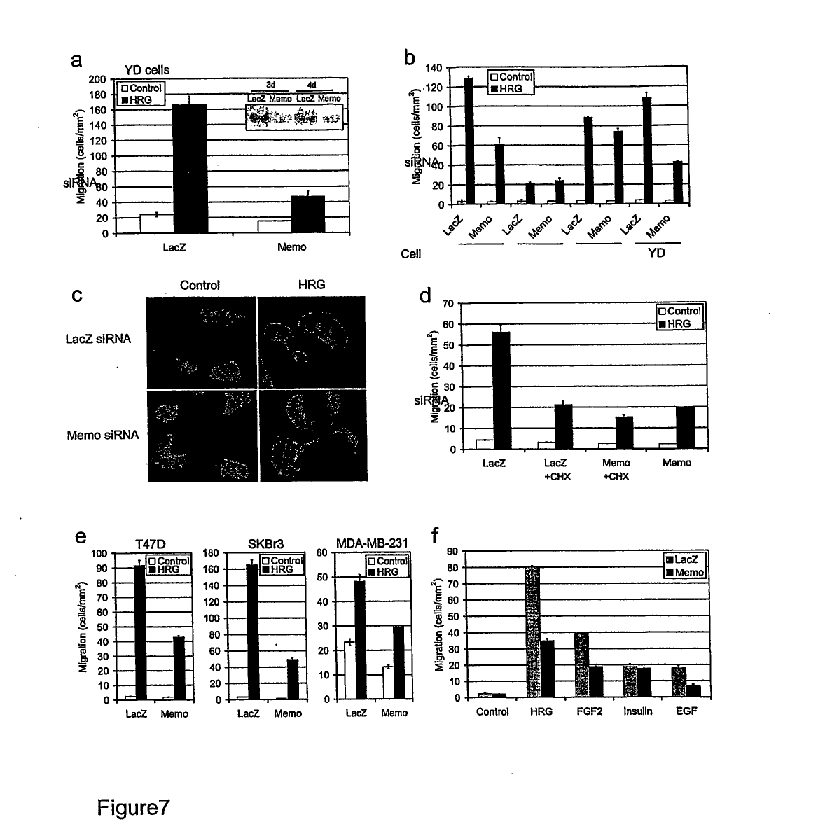

The function of CGI-27, the hypothetical protein identified as a specific phospho-YD binder, was until now unknown. Furthermore, its sequence does not provide any information on a potential role in migration. The inventors tested CGI-27 function using specific siRNA to knock-down its expression. Quantitative PCR revealed that CGI-27 mRNA expression was around 80% lower in siRNA transfected cells relative to control cells (FIG. 7a, insert). Importantly, loss of CGI-27 strongly decreased migration of YD cells in response to HRG (FIG. 7a). Based upon these and the following results, CGI-27 was named Memo for mediator of ErbB2-dependent cell motility.

Inhibition of Memo's expression had a strong effect on HRG-induced migration of YD, but not YC cells, demonstrating that Memo acts specifically downstream of the YD tyrosine residue (FIG. 7b). Importantly, HRG-induced formation of lamellipodia was not affected by a reduction in Memo levels (FIG. 7c), showing that, in contrast to the other identified signalling molecules, Memo is not involved in early steps of cell migration. In addition, Memo siRNA, did not block migration of NYPD-expressing cells (FIG. 7b), whose migration is essentially dependent on post-translational events (FIG. 4). Examining the effects of cycloheximide treatment and Memo knock-down on YD cells revealed that migration was blocked to a similar extent by both approaches and that their effects were not additive (FIG. 7d), suggesting that Memo depends on de novo protein synthesis to stimulate cell migration. The inventors have therefore provided evidence that Memo is a signalling molecule that links phosphorylated YD to late stages of cell migration, independent of cytoskeletal actin reorganization.

### Memo is Required for ErbB2-Dependent Microtubule Outgrowth

Formation of lamellipodia is dependent on the formation of actin filaments. Accordingly, Inhibition of Memo's expression through siRNA targeting did not prevent the formation of actin fibers.

Recent studies demonstrate the central role of the microtubule cytoskeleton for cell polarity and cell migration²⁷. HRG induced the extension of microtubule from the centrosome to the cell periphery in T47D cells. However, when Memo's expression was inhibited, the network of microtubules induced by HRG was severely reduced in T47D and SKBr3 cells. Quantitation revealed that the number of T47D and SKBr3 cells showing microtubule outgrowth was reduced from around 80% in control cells to 20% in cells transfected with Memo siRNA. Interestingly, the perinuclear microtubule network, which is not dependent on HRG stimulation, was not affected by the decrease in Memo's expression. This data show that Memo is required for the ErbB2-dependent elongation of microtubules to the cell cortex.

In summary, Memo is not involved in stages of cell migration, such as lamellipodia formation, which are linked to remodeling of the actin cytoskeleton—nevertheless it is required for the extension of a microtubule network to the cell periphery. Microtubules grow out from the centrosome, their plus ends exploring the cytoplasm through alternate phases of growth and shortening, a phenomenon termed dynamic instability. Microtubule dynamics can be modulated by two types of molecules: microtubule-associated proteins, such as MCAK (mitotic centromere-associated kinesins), which bind to microtubule ends and destabilize them; and plus-end binding proteins that favor microtubule growth by binding the growing end, allowing microtubules to reach their target destination. HRG triggers the growth of microtubules from the centrosome to the cell cortex and this is prevented in the absence of Memo. Thus, Memo could be a linker between extracellular chemotactic cues and the microtubule cytoskeleton, allowing the stabilization of outgrowing microtubules and the maintenance of cell polarity by preventing microtubule destabilization or promoting microtubule extension. Interestingly, Memo is not required for the organization or maintenance of the central microtubules, indicating a specific role for Memo in stabilizing the most dynamic microtubule extending toward the protruding membrane of migrating cells. Microtubules are also a key element for cell division. The fact that Memo is not required for microtubule organization in general, but specifically for microtubule outgrowth within lamellipodia, can explain why knocking down Memo's expression does not interfere with breast carcinoma cell proliferation.

Both T47D and SKBr3 cells are capable of extending polarized lamellipodia in the absence of microtubule outgrowth. Similarly, nocodazole-treated cells do not grow microtubules, but are still capable of forming lamellipodia indicating that microtubule outgrowth is not required for early actin cytoskeleton remodeling. However, we have observed that in cells lacking Memo, while microtubule outgrowth is inhibited, the amount of actin stress fibers appears to be increased, which highlights the dynamic interactions that take place between the actin and the microtubule cytoskeleton in migrating cells.

### Memo is a General Mediator of Breast Carcinoma Cell Motility

The present data demonstrates that Memo is required for migration of YD cells. HRG-induced migration of Neu expressing cells (FIG. 7b), and parental ErbB2-expressing T47D cells, (FIG. 7e), was also 50% dependent on Memo expression. Thus, in the context of the wild type receptor, YC-dependent signalling is not able to fully offset the loss of functional Memo.

The SKBr3 and MDA-MB-231 cell lines are frequently used as experimental breast tumor models. Due to its high expression in SKBr3 cells, ErbB2 is activated and promotes constitutive signaling of the MAPK and PI3K pathways^{28,29}. Despite this, SKBr3 cells display only low basal migration. In contrast, MDA-MB-231 cells are highly motile in the absence of ligands and display metastatic growth in animal models. SKBr3 and MDA-MB-231 cells with knocked-down Memo showed decrease in HRG-induced migration (FIG. 7e). Reduction in Memo expression also lowered basal migration of MDA-MB-231 cells (FIG. 7e), which right reflect the presence of autocrine activated ErbB2 in these cells^{9,30}.

The inventors also examined the role of Memo downstream of other tyrosine kinase receptors. Fibroblast growth factor (FGF) 2 and, to a lower degree, insulin and epidermal growth factor (EGF) stimulated T47D cell migration (FIG. 7f). SiRNA-mediated knock-down of Memo did not affect insulin-dependent migration, but strongly reduced FGF2- and EGF-induced cell migration (FIG. 7f), indicative of a widespread role for Memo in cell motility, for example in wound healing, angiogenesis (e.g., in tumour growth) and inflammation.

### REFERENCES

1. Varis, A. et al. Targets of gene amplification and overexpression at 17q in gastric cancer. Cancer Res 62, 2625-9 (2002).
2. Slamon, D. J. et al. Studies of the HER-2/neu proto-oncogene in human breast and ovarian cancer. Science 244, 707-12 (1989).
3. Slamon, D. J. et al. Human breast cancer: correlation of relapse and survival with amplification of the HER-2/neu oncogene. Science 235, 177-82 (1987).
4. Dankort, D. et al. Grb2 and Shc adapter proteins play distinct roles in Neu (ErbB-2)-induced mammary tumorigenesis: implications for human breast cancer. Mol Cell Biol 21, 1540-51 (2001).
5. Guy, C. T., Cardiff, R. D. & Muller, W. J. Activated neu induces rapid tumor progression. J Biol Chem 271, 7673-8 (1996).
Guy, C. T. et al. Expression of the neu protooncogene in the mammary epithelium of transgenic mice induces metastatic disease. Proc Natl Acad Sci USA 89, 10578-82 (1992).
7. Adam, L. et al. Heregulin regulates cytoskeletal reorganization and cell migration through the p21-activated kinase-1 via phosphatidylinositol-3 kinase. J Biol Chem 273, 28238-46 (1998).
8. Spencer, K. S.; Graus-Porta, D., Leng, J., Hynes, N. E. & Klemke, R. L. ErbB2 is necessary for induction of carcinoma cell invasion by ErbB family receptor tyrosine kinases. J Cell Biol 148, 385-97 (2000).
9. Hijazi, M. M. et al. Heregulin regulates the actin cytoskeleton and promotes invasive properties in breast cancer cell lines. Int j Oncol 17, 629-41 (2000).
10. Xu, F. J. et al. Heregulin and agonistic anti-p185 (c-erbB2) antibodies inhibit proliferation but increase invasiveness of breast cancer cells that overexpress p185 (c-erbB2): increased invasiveness may contribute to poor prognosis. Clin Cancer Res 3, 1629-34 (1997).
11. Segatto, O., Lonardo, F., Pierce, J. H., Bottaro, D. P. & Di Fiore, P. P. The role of autophosphorylation in modulation of erbB-2 transforming function. New Biol 2, 187-95 (1990).
12. Hazan, R. et al. Identification of autophosphorylation sites of HER2/neu. Cell Growth Differ 1, 3-7 (1990).
13. Dankort, D. L., Wang, Z., Blackmore, V., Moran, M. F. & Muller, W. J. Distinct tyrosine autophosphorylation sites negatively and positively modulate neu-mediated transformation. Mod Cell Biol 17, 5410-25 (1997).
14. Segatto, O. et al. Shc products are substrates of erbB-2 kinase. Oncogene 8, 2105-12 (1993).
15. Hellyer, N. J., Kim, M. S. & Koland, J. G. Heregulin-dependent activation of phosphoinositide 3-kinase and Akt via the ErbB2/ErbB3 co-receptor. J Biol Chem 276, 42153-61 (2001).
16. Pawson, T. Protein modules and signalling networks. Nature 373, 573-80 (1995).
17. Yarden, Y. & Sliwkowski, M. X. Untangling the ErbB signalling network. Nat Rev Mol Cell Biol 2, 127-37 (2001).
18. Schlessinger, J. Cell signaling by receptor tyrosine kinases. Cell 103, 211-25 (2000).
19. Kavanaugh, W. M., Turck, C. W. & Williams, L. T. PTB domain binding to signaling proteins through a sequence motif containing phosphotyrosine. Science 268, 1177-9 (1995).
20. Klemke, R. L. et al. Regulation of cell motility by mitogen-activated protein kinase. J Cell Biol 137, 481-92 (1997).
21. Keely, P. J., Westwick, J. K., Whitehead, I. P., Der, C. J. & Parise, L. V. Cdc42 and Rac1 induce integrin-mediated cell motility and invasiveness through PI(3)K. Nature 390, 632-6 (1997).
22. Zrihan-Licht, S. et al. RAFTK/Pyk2 tyrosine kinase mediates the association of p190 RhoGAP with RasGAP and is involved in breast cancer cell invasion. Oncogene 19, 1318-28 (2000).
23. Vadlamudi, R., Adam, L., Talukder, A., Mendelsohn, J. & Kumar, R. Serine phosphorylation of paxillin by heregulin-beta1: role of p38 mitogen activated protein kinase. Oncogene 18, 7253-64 (1999).
24. Beerli, R. R., Wels, W. & Hynes, N. E. Intracellular expression of single chain antibodies reverts ErbB-2 transformation. J Biol Chem 269, 23931-6 (1994).
25. Graus-Porta, D., Beerli, R. R. & Hynes, N. E. Single-chain antibody-mediated intracellular retention of ErbB-2 impairs Neu differentiation factor and epidermal growth factor signaling. Mol Cell Biol 15, 1182-91 (1995).
26. Hall, A. Rho GTPases and the actin cytoskeleton. Science 279, 509-14 (1998).
27. Dankort, D., Jeyabalan, N., Jones, N., Dumont, D. J. & Muller, W. J. Multiple ErbB-2/Neu Phosphorylation Sites Mediate Transformation through Distinct Effector Proteins. J Biol Chem 276, 38921-8 (2001.).
28. Neve, R. M. et al. Effects of oncogenic ErbB2 on G1 cell cycle regulators in breast tumour cells. Oncogene 19, 1647-56 (2000).
29. Moasser, M. M., Basso, A., Averbuch, S. D. & Rosen, N. The tyrosine kinase inhibitor ZD1839 (“Iressa”) inhibits HER2-driven signaling and suppresses the growth of HER2-overexpressing tumor cells. Cancer Res 61, 7184-8 (2001).
30. Tsai, M. S., Shamon-Taylor, L. A., Mehmi, I., Tang, C. K. & Lupu, R. Blockage of heregulin expression inhibits tumorigenicity and metastasis of breast cancer. Oncogene 22, 761-8 (2003).
31. Lane, H. A. et al. ErbB2 potentiates breast tumor proliferation through modulation of p27 (Kip1)-Cdk2 complex formation: receptor overexpression does not determine growth dependency. Mol Cell Biol 20, 3210-23 (2000).
32. Ranson, M. & Slikowski, M. X Perspectives on anti-HER monoclonal antibodies. Oncology 63 Suppl 1, 17-24 (2002).
33. Malliri, A. et al. The transcription factor AP-1 is required for EGF-induced activation of rho-like GTPases, cytoskeletal rearrangements, motility, and in vitro invasion of A431 cells. J Cell Biol 143, 1087-99 (1998).
34. Jauliac, S. et al. The role of NFAT transcription factors in integrin-mediated carcinoma invasion. Nat Cell Biol 4, 540-4 (2002).
35. Kustikova, O. et al. Fra-1 induces morphological transformation and increases in vitro invasiveness and motility of epithelioid adenocarcinoma cells. Mol Cell Biol 18, 7095-105 (1998).
36. Lai, C. H., Chou, C. Y., Ch'ang, L. Y., Liu, C. S. & Lin, W. Identification of novel human genes evolutionarily conserved in Caenorhabditis elegans by comparative proteomics. Genome Res 10, 703-13 (2000).
37. Keely, P. J., Fong, A. M., Zutter, M. M. & Santoro, S. A. Alteration of collagen-dependent adhesion, motility, and morphogenesis by the expression of antisense alpha 2 integrin mRNA in mammary cells. J Cell Sci 108 (Pt 2), 595-607 (1995).
38. Sander, E. E. et al. Matrix-dependent Tiam1/Rac signaling in epithelial cells promotes either cell-cell adhesion or cell migration and is regulated by phosphatidylinositol 3-kinase. J Cell Biol 143, 1385-98 (1998).
39. Cho, S. Y. & Klemke, R. L. Extracellular-regulated kinase activation and CAS/Crk coupling regulate cell migration and suppress apoptosis during invasion of the extracellular matrix. J Cell Biol 149, 223-36 (2000).
40. Kisielow, M., Kleiner, S., Nagasawa, M., Faisal, A. & Nagamine, Y. Isoform-specific knockdown and expression of adaptor protein ShcA using small interfering RNA. Biochem J 363, 1-5 (2002).
41. Cappellen, D. et al. Transcriptional program of mouse osteoclast differentiation governed by the macrophage colony-stimulating factor and the ligand for the receptor activator of NFkappa B. J Biol Chem 277, 21971-82 (2002).

### Sequence Annex I—Genbank AF132961

| | |
|---|---|
| LOCUS | AF132961 1553 bp mRNA |
| | linear PRI 18 MAY 2000 |
| DEFINITION | Homo sapiens CGI-27 protein mRNA, complete cds. |
| ACCESSION | AF132961 |
| VERSION | AF132961.1 GI:4680692 |
| KEYWORDS | . |
| SOURCE | Homo sapiens (human) |
| ORGANISM | Homo sapiens |
| | Eukaryota; Metazoa; Chordata; Craniata; Vertebrata; |
| | Euteleostomi; Mammalia; Eutheria; Primates; |
| | Catarrhini; Hominidae; Homo. |
| REFERENCE | 1 (bases 1 to 1553) |
| AUTHORS | Lai, C. H., Chou, C. Y., Ch'ang, L. Y., |
| | Liu, C. S. and Lin, W. |
| TITLE | Identification of novel human genes evolutionarily |
| | conserved in Caenorhabditis elegans by |
| | comparative proteomics |
| JOURNAL | Genome Res. 10 (5), 703-713 (2000) |
| MEDLINE | 20272150 |
| PUBMED | 10810093 |
| REFERENCE | 2 (bases 1 to 1553) |
| AUTHORS | Lin, W. -C. |
| TITLE | Direct Submission |
| JOURNAL | Submitted (4 Mar. 1999) Institute of Biomedical |
| | Sciences, Academia |
| | Sinica, No. 128, Sec. II, Academia Road, Taipei 115, |
| | Taiwan |

| FEATURES | Location/Qualifiers |
|---|---|
| source | 1..1553 |
| | /organism=“Homo sapiens” |
| | /db_xref=“taxon:9606” |
| CDS | 116..1009 |
| | /codon_start=1 |
| | /product=“CGI-27 protein” |
| | /protein_id=“AAD27736.1” |
| | /db_xref=“GI:4680693” |

| /translation = “MSNRVVCREASHAGSWYTASGPQLNAQLEGWLSQVQSTKRPARA | |
|---|---|
| IIAPHAGYTYCGSCAAHAYKQVDPSITRRIFILGPSHHVPLSRCALSSVDIYRTPLYD | |
| LRIDQKIYGELWKTGMFERMSLQTDEDEHSIEMHLPYTAKAMESHKDEFTIIPVLVGA | |
| LSESKEQEFGKLFSKYLADPSNLFVVSSDFCHWGQRFRYSYYDESQGEIYRSIEHLDK | |
| MGMSIIEQLDPVSFSNYLKKYHNTICGRHPIGVLLNAITELQKNGMNMSFSFLNYAQS | |
| SQCRNWQDSSVSYAAGALTVH” | |
| BASE COUNT 445 a 336 c 330 g 442 t | |
| ORIGIN | |
| 1 | tgccgcctcc tcctgggctg ggcgcggtgt ctcgtcccct cgcggagcgc |
| | tcctgccgcc |
| 61 | gccgccgccg cctcctcatt catcctcgtg caccataggc ggcacaggca |
| | ccaagatgtc |
| 121 | caaccgagtg gtctgccgag aagccagtca cgccgggagc tggtacacag |
| | cctcaggacc |
| 181 | gcagctgaat gcacagctag aaggttggct ttcacaagta cagtctacaa |
| | aaagacctgc |
| 241 | tagagccatt attgcccccc atgcaggata tacgtactgt gggtcttgtg |
| | ctgcccatgc |
| 301 | ttataaacaa gtggatccgt ctattacccg gagaattttc atccttgggc |
| | cttctcatca |
| 361 | tgtgcccctc tctcgatgtg cactttccag tgtggatata tataggacac |
| | ctctgtatga |
| 421 | ccttcgtatt gaccaaaaga tttacggaga actgtggaag acaggaatgt |
| | ttgaacgcat |
| 481 | gtctctgcag acagatgaag atgaacacag tattgaaatg catttgcctt |
| | atacagctaa |
| 541 | agccatggaa agccataagg atgagtttac cattattcct gtactggttg |
| | agctctgag |
| 601 | tgagtcaaaa gaacaggaat tcggaaaact cttcagtaaa tatctagcgg |
| | atcctagtaa |
| 661 | tctctttgtg gtttcttctg atttctgcca ttggggtcaa aggttccgtt |
| | acagttacta |
| 721 | tgatgaatcc cagggggaga tttatagatc cattgaacat ctagataaaa |
| | tgggtatgag |
| 781 | tattatagaa caattagacc ctgtatcttt tagcaattac ttgaagaaat |
| | accataatac |
| 841 | tatatgtgga agacatccca ttggggtgtt attaaatgct atcacagagc |
| | tccagaagaa |
| 901 | tggaatgaat atgagttttt cgtttttgaa ttatgcccag tcgagccagt |
| | gtagaaactg |
| 961 | gcaagacagt tcagtgagtt atgcagctgg agcactcacg gtccactgaa |
| | gctctgaatc |
| 1021 | ctcagggatg ccacctgcac attctcatac tctgtccggg gtcccagcct |
| | agcctttacc |
| 1081 | acgatactgg tcctggtttg gggggattct gaaacctcaa actaatagaa |
| | ctttcttctc |
| 1141 | tttttttcta gtaggtgtag tccttcctta atttcaactc attaaaaaat |
| | gctttatagt |
| 1201 | ttagggcagt ggaaggaagg ctggcatcaa aatattttga tcaaaaaaga |
| | tgacaatgta |
| 1261 | aaggctcagt tgtggcagac agttttttga aagtaacttg taaagcattt |
| | accatatcct |
| 1321 | aaatttgcac tctttgcaga cttgtgcaca tatattccgc tttcagaata |
| | gttttgcaaa |
| 1381 | ttgtacacaa acaaacaaaa aggtggaagc tttttaataa agaaattgca |
| | tttataaatg |
| 1441 | atctgtatta gaatataata aatctccagt tatagtcaat tactacccat |
| | gttgtacaac |
| 1501 | agataccttc tattttagtt gctaataaag ggctacacaa ctcaaaaaaa |
| | aaa |
| | // |

Revised: Jul. 5, 2002.

1. A method of modulating the ability of a cell to migrate, which method comprises modulating an activity of MEMO, or a MEMO homologue, in the cell, wherein MEMO has the amino acid sequence set out in Sequence Annex 1.

2. A method as claimed in claim 1 wherein the ability of the cell to carry out a late stage of migration is modulated

3. A method as claimed in claim 2 wherein the ability of the cell to carry out a late stage of migration is preferentially modulated over the ability to carry out an early stage of migration.

4. A method as claimed in claim 1 wherein the modulation is inhibition.

5. A method as claimed in claim 1 wherein the ability of the cell to migrate is in response to a migration-inducing signal.

6. A method as claimed in claim 5 wherein the migration-inducing signal is a signal from the ErbB2 receptor.

7. A method as claimed in claim 5 wherein the migration-inducing signal is a signal from the Fibroblast Growth factor (FGF) 2 receptor or the Epidermal Growth Factor (EGF) receptor.

8. A method as claimed in claim 6 wherein the migration-inducing signal results from contacting said receptor with a ligand.

9. A method as claimed in claim 4 wherein the inhibition of activity is caused by down-regulating MEMO, or the MEMO homologue, in the cell.

10. A method as claimed in claim 9 wherein the down-regulation is caused by siRNAs.

11. A method as claimed in claim 4 wherein the inhibition of activity is caused by inhibiting the interaction of MEMO, or the MEMO homologue, with a binding partner in the cell.

12. A method as claimed in claim 11 wherein the binding partner is ErbB2.

13. A method as claimed in claim 11 wherein the binding partner is she.

14. A method as claimed in claim 11 wherein the inhibition of the interaction is caused by an inhibitor selected from: an antibody or antibody fragment.

15. A method for identifying a substance which modulates cell migration, the method comprising determining if the substance binds to and/or modulates an activity of a MEMO polypeptide selected from: MEMO, or a variant or fragment thereof.

16. A method as claimed in claim 15 wherein the modulation is inhibition.

17. A method as claimed in claim 16 wherein the inhibition is of a late stage of migration preferentially over an early stage of migration.

18. A method as claimed in claim 16 which comprises the steps of:
(i) contacting a cell expressing MEMO, or a variant thereof which has the ability to mediate cell migration, with a test substance, and
(ii) identifying substances which inhibit an activity of MEMO, or the variant, in the cell.

19. A method as claimed in claim 16 which comprises the steps of:
(i) contacting the MEMO polypeptide with a binding partner in the presence and absence of a test substance;
(ii) determining whether the presence of a test substance inhibits the interaction between the MEMO polypeptide and the binding partner.

20. A method as claimed in claim 19 wherein the MEMO polypeptide or the binding partner is labelled with a detectable label, and the other is immobilized on a solid support.

21. A method as claimed in claim 20 which comprises:
(i) providing a cell capable of expressing the MEMO polypeptide and its binding partner and a reporter gene construct,
(ii) contacting the cell with a test substance,
whereby inhibition by the test substance of binding between the MEMO polypeptide and the binding partner can be observed as a reduction of reporter gene expression.

22. A method as claimed in claim 19 wherein the variant or fragment of MEMO has the ability to mediate cell migration

23. A method as claimed in claim 19 wherein the fragment of MEMO is at least 20, 30, 40, 50, 75, 100, 150 or more amino acids in size.

24. A method as claimed in claim 18 wherein the binding partner is provided by the method of claim 50.

25. A method as claimed in claim 19 wherein the binding partner is an upstream factor.

26. A method as claimed in claim 25 wherein the upstream factor is ErbB2 or a fragment thereof.

27. A method as claimed in claim 26 comprising the steps of:
(i) providing an ErbB2 polypeptide which is ErbB2, or a fragment thereof, comprising a phosphorylated residue corresponding to Y1227;
(ii) contacting said ErbB2 polypeptide with the MEMO polypeptide, in the presence and absence of a test substance;
(iii), determining whether the presence or absence of a test substance inhibits the interaction between the ErbB2 polypeptide and MEMO polypeptide.

28. A method as claimed in claim 27 wherein step (iii) is carried out by determining whether the test substance inhibits chemical modification of the MEMO polypeptide by the ErbB2 polypeptide.

29. A method as claimed in claim 27 wherein the step (iii) is carried out by determining whether the test substance inhibits the physical association between MEMO and the ErbB2 polypeptide.

30. A method as claimed in claim 25 wherein the upstream factor is she or a fragment thereof.

31. A method as claimed in claim 15 further comprising the step of confirming that the substance inhibits migration of a cell in response to a migration-inducing signal.

32. An isolated MEMO polypeptide comprising the amino acid sequence set out in Sequence Annex I.

33. An isolated polypeptide which is a variant of the MEMO polypeptide of claim 1, having at least 50%, 60%, 70%, 80%, 90%, 95% or 99% amino acid sequence identity thereto

34. An isolated polypeptide which is a variant as claimed in claim 33 which has the ability to mediate cell migration.

35. A vector comprising a nucleic acid having a MEMO polynucleotide sequence encoding the polypeptide of claim 32.

36. A vector as claimed in claim 35 wherein the MEMO polynucleotide sequence is that set out in Sequence Annex I.

37. A vector as claimed in claim 35 which is an expression vector comprising a promoter operably linked to said nucleic acid.

38. A process for preparing a polypeptide, which process comprises cultivating a host cell transformed or transfected with an expression vector as claimed in claim 37 under conditions to provide for expression by the vector of said nucleic acid encoding the polypeptide, and recovering the expressed polypeptide.

39. A host cell which expresses a heterologous polypeptide of claim 32.

40. A nucleic acid having a polynucleotide sequence which is complementary to the MEMO polynucleotide described in claim 36.

41. Double-stranded RNA which comprises an RNA sequence encoding MEMO, a MEMO homologue, or a fragment thereof, wherein MEMO has the amino acid sequence set out in Sequence Annex I.

42. Double-stranded RNA as claimed in claim 41 which is a siRNA duplex consisting of between 20 and 25 bps.

43. A siRNA duplex as claimed in claim 42 wherein the RNA sequence encoding MEMO, or a MEMO homologue, or the fragment thereof, corresponds to positions 460-480 of the MEMO polynucleotide sequence set out in Sequence Annex I.

44. A vector encoding the dsRNA or siRNA duplex as claimed in claim 41.

45. A method of producing the siRNA duplex of claim 42, the method comprising introducing the vector of claim 44 into a host cell and causing or allowing transcription from the vector in the cell.

46. A method of producing the siRNA duplex of claim 42, the method comprising introducing,
(i) a vector encoding the sense sequence of the siRNA duplex, and
(ii) a vector encoding the anti-sense sequence of the siRNA duplex, into a host cell and causing or allowing transcription from the vectors in the cell.

47. A method for producing a transgenic non-human mammal in which the ability of a cell to migrate is inhibited, the method comprising incorporating a lesion into the locus of a MEMO homologue therein, wherein MEMO has the amino acid sequence set out in Sequence Annex I.

48. A transgenic non-human animal in which expression of a MEMO homologue, is modified such as to modulate the ability of a cell therein to migrate, wherein MEMO has the amino acid sequence set out in Sequence Annex I.

49. An isolated antibody which binds specifically to MEMO, wherein MEMO has the amino acid sequence set out in Sequence Annex I.

50. A method of identifying a binding partner of MEMO, which method comprises:
(i) providing a MEMO polypeptide selected from: MEMO, or a variant or fragment thereof,
(ii) contacting the MEMO polypeptide with the putative MEMO binding partner,
(iii) determining whether the MEMO binding partner is able to bind to the MEMO polypeptide,
wherein MEMO has the amino acid sequence set out in Sequence Annex I.

51. A method as claimed in claim 50 wherein the variant or fragment of MEMO has the ability to mediate cell migration

52. A method as claimed in claim 40 wherein the fragment of MEMO is at least 20, 30, 40, 50, 75, 100, 150 or more amino acids in size.

53. A method as claimed in claim 50 wherein the MEMO polypeptide is provided in an activated form.

54. A method as claimed in claim 50 wherein the MEMO polypeptide is immobilized on a solid support and the immobilized MEMO polypeptide is contacted with the putative MEMO binding partner.

55. A method as claimed in claim 54 wherein the immobilized MEMO polypeptide is:
(i) contacted with a sample which contains multiple putative binding partners,
(ii) unbound material is washed away,
(iii) material bound to the immobilized MEMO polypeptide is released, and
(iv) the identity of protein bound to the immobilized MEMO polypeptide is then assessed.

56. A method as claimed in claim 50 which comprises providing a cell capable of expressing the MEMO polypeptide, and its putative binding partner, and a reporter gene construct, whereby binding between the MEMO polypeptide and the binding partner can be observed by reporter gene expression.

57. A method of identifying a mediator of cell migration, which method comprises performing a method as claimed in claim 50, and confirming that the MEMO binding partner is a mediator of cell migration.

58. A method as claimed in claim 57 wherein the MEMO binding partner is a specific mediator of late stage over early stage cell migration.

59. A method as claimed in claim 58 comprising the steps of:
(i) providing a cell in which the activity of the MEMO binding partner is modulated;
(ii) detecting whether early stage migration events are affected by said modulation;
(iii) detecting whether late stage migration events are affected by said modulation.

60. A method as claimed in claim 56 wherein the modulation is inhibition.

61. A method as claimed in claim 58 comprising the step of determining whether the MEMO binding partner is required for signal transduction from ErbB2 residues Y1201 or Y1227.

62. A method as claimed in claim 61 comprising the steps of
i) providing a first cell in which ErB2-induced cell migration signalling is mediated by Y1227 and not Y1201;
ii) providing a second cell in which ErbB2-induced cell migration signalling is mediated by Y1201 and not Y1227;
iii) inhibiting the activity of the MEMO binding partner of interest in said cells.

63. A process for producing a medicament for the treatment of a patient suffering from a disease in which it is desired to control cell motility, the process comprising formulating an inhibitor of MEMO activity as the medicament, wherein MEMO has the amino acid sequence set out in Sequence Annex I.

64. An inhibitor of MEMO activity for use in the treatment of a patient suffering from a disease in which it is desired to control cell motility.

65. Use of an inhibitor of MEMO activity in the preparation of a medicament for the treatment of a patient suffering from a disease in which it is desired to control cell motility.

66. A process, inhibitor or use as claimed in claim 63 wherein the inhibitor is provided by the method of claim 16.

67. A process, inhibitor or use as claimed in claim 63 wherein the treatment is to prevent metastasis and or angiogenesis in cancer.

68. A process, inhibitor or use as claimed in claim 63 wherein the treatment comprises use of a method as claimed in claim 4 which method comprises inhibiting the ability of a cell in the patient to migrate by inhibiting an activity of MEMO, or a MEMO homologue, in the cell.

69. A process, inhibitor or use as claimed in claim 68 wherein the cell is a tumour cell or other cell implicated in cancer or other metastatic disease.

70. A method as claimed in claim 1 for the treatment of a patient suffering from a disease in which it is desired to promote cell motility, which method comprises promoting the ability of a cell in the patient to migrate by promoting an activity of MEMO, or a MEMO homologue, in the cell.

71. A method as claimed in claim 70 for promoting wound healing.
